(19) United States
(12) Patent Application Publication
(10) Pub. No.: US 2015/0238532 A1
(43) Pub. Date: Aug. 27, 2015

(54) METHODS TO ISOLATE HUMAN MESENCHYMAL STEM CELLS

(71) Applicant: ALBERT EINSTEIN COLLEGE OF MEDICINE OF YESHIVA UNIVERSITY, Bronx, NY (US)

(72) Inventors: Paul S. Frenette, New York, NY (US); Sandra Pinho, Bronx, NY (US); Julie Lacombe, Bronx, NY (US)

(73) Assignee: ALBERT EINSTEIN COLLEGE OF MEDICINE OF YESHIVA UNIVERSITY, Bronx, NY (US)

(21) Appl. No.: 14/416,078

(22) PCT Filed: Jul. 1, 2013
(86) PCT No.: PCT/US13/48851

§ 371 Date: Jan. 21, 2015

Related U.S. Application Data
(60) Provisional application No. 61/675,462, filed on Jul. 25, 2012.

(51) Int. Cl. A61K 35/28; C12N 5/0775

(52) U.S. Cl. CPC A61K 35/28; C12N 5/0663

(57) ABSTRACT

A method of obtaining a population of PDGFRα⁺ CD51⁺ CD146^{high} stem cells is provided. Compositions comprising PDGFRα⁺ CD51⁺ CD146^{high} stem cells, and methods of use of a population of PDGFRα⁺CD51⁺ CD146^{high} stem cells, are also provided.

### CROSS-REFERENCE TO RELATED APPLICATIONS

This application claims benefit of U.S. Provisional Application No. 61/675,462, filed Jul. 25, 2012, the contents of which are hereby incorporated by reference.

### STATEMENT OF GOVERNMENT SUPPORT

This invention was made with government support under grant numbers R01DK056638 and R01HL097819 awarded by the National Institutes of Health. The government has certain rights in the invention.

### BACKGROUND OF THE INVENTION

The disclosures of all publications, including as referred to herein by name and year in parentheses, and the disclosures of all patents, patent application publications and books referred to in this application, are hereby incorporated by reference in their entirety into the subject application to more fully describe the art to which the subject invention pertains.

Hematopoietic stem cells (HSCs) continuously replenish all blood cell lineages throughout lifetime. Incipient hematopoiesis is first detected extra-embryonically in the yolk sac, and later in the aorta-gonad-mesonephros region from where it moves transiently to the placenta and liver before being stabilized in the fetal bone marrow (Wang and Wagers, 2011). In the adult stage, HSCs reside in the highly complex and dynamic microenvironment of the bone marrow now commonly referred to as HSC niche (Schofield, 1978). The interactions between the niche constituents and HSCs ensure hematopoietic homeostasis by regulating HSCs self-renewal, differentiation and migration and by integrating neural and hormonal signals from the periphery (Mendez-Ferrer et al., 2009; Mendez-Ferrer et al., 2010; Mercier et al., 2012; Wang and Wagers, 2011).

The cellular constituents of the HSC niche and their role are still poorly understood; however, in the last decade, several putative cellular components of the murine HSC niche have been proposed, including osteoblastic, endothelial, adipocytic and perivascular cells (Arai et al., 2004; Calvi et al., 2003; Chan et al., 2009; Ding et al., 2012; Kiel et al., 2005; Mendez-Ferrer et al., 2010; Naveiras et al., 2009; Sugiyama et al., 2006; Zhang et al., 2003). Multipotent bone marrow mesenchymal stem cells (MSCs) have long been proposed to also provide regulatory signals to hematopoietic progenitors, as mixed cultures derived from the adherent fraction of the bone marrow stroma promotes the maintenance of HSCs in vitro (Dexter et al., 1977). The prospective identification and functional characterization of naïve populations of mouse and/or human bone marrow stromal MSCs has been mired by the absence of specific cell surface markers allowing prospective isolation. Several MSC-associated antigens have been proposed (such as CD31⁻ CD34⁻ CD45⁻ CD105⁺ CD90⁺ CD73⁺) (Dominici et al., 2006) in cultured cells. Nevertheless, these markers are not homogeneously expressed across cultures, varying with isolation protocols and passage, therefore not necessarily representative of MSCs in vivo. Very few MSC-associated antigens have been validated using rigorous transplantation assays (Mendez-Ferrer et al., 2010; Sacchetti et al., 2007). In the mouse bone marrow, the expression of the intermediate filament protein Nestin, characterizes a rare population of multipotent MSCs in close contact with the vasculature and HSCs. Nestin⁺ stromal cells contain all the fibroblastic colony-forming units (CFU-F) activity within the mouse bone marrow and the exclusive capacity to form clonal non-adherent spheres in culture (Mendez-Ferrer et al., 2010). The selective ablation of mouse Nestin⁺ cells (Mendez-Ferrer et al., 2010) or CXCL12-abundant reticular (CAR) cells (Omatsu et al., 2010) led to significant alterations in bone marrow HSC and progenitor maintenance, respectively. Serial transplantation analyses revealed that Nestin⁺ cells are able to self-renew and generate hematopoietic activity in heterotopic bone ossicle assays (Mendez-Ferrer et al., 2010). This potential was also associated with a CD45⁻ Tie2⁻ CD51⁺ CD105⁺ CD90⁻ subset from the fetal mouse bone (Chan et al., 2009). However, in the human bone marrow, MSCs are still retrospectively isolated based on plastic adherence (Friedenstein et al., 1970; Pittenger et al., 1999). Human CD45⁻ CD146^{high} self-renewing osteoprogenitors isolated from stromal cultures were shown capable of generating a heterotopic bone marrow niche in a subcutaneous transplantation model, containing all the human bone marrow CFU-F activity (Sacchetti et al., 2007). However, a recent study showed that human CD45⁻ CD271⁺ CD146^{-/low} bone marrow cells also possess these capacities (Tormin et al., 2011).

Since Nestin is an intracellular protein, its identification in non-transgenic mice and humans requires cell permeabilization which precludes prospective isolation of live cells.

The present invention addresses the need for a specifically identifiable and isolatable population of HSCs, and also provides methods of isolation thereof and use thereof, and the need for identifying a combination of surface markers defining Nestin+ cells that can be used to isolate Nestin+ MSCs able to support HSC expansion in vitro.

### SUMMARY OF THE INVENTION

This invention provides a method of obtaining a population of stem cells comprising identifying PDGFRα⁺ CD51⁺ cells in a population of cells, and recovering the PDGFRα⁺ CD51⁺ cells so as to obtain the population of stem cells.

This invention also provides a method of obtaining a population of stem cells comprising identifying PDGFRα⁺ CD51⁺ cells in a population of cells, and separating the PDGFRα⁺ CD51⁺ (αV integrin⁺) cells and recovering the PDGFRα⁺ CD51⁺ cells so as to obtain the population of stem cells.

Also provided is an isolated population of PDGFRα⁺ CD51⁺ (αV integrin⁺) mesenchymal stem cells, wherein the population is 50% or greater PDGFRα⁺ CD51⁺ cells.

Also provided is a method comprising co-culturing a population of cells comprising stem cells with any of the above described PDGFRα⁺ CD51⁺ cells, or populations of such cells, so as to produce an expanded population of stem cells.

Also provided is a composition comprising any of the above-described PDGFRα⁺ CD51⁺ cells, or populations of such cells, and a carrier.

Also provided is a method comprising administering an amount of any of the described populations of stem cells, or the described compositions, to a subject, in an amount effective to confer stem cell activity on a subject.

Also provided is a method of enhancing hematopoietic activity in a subject comprising administering an amount of (i) the population of stem cells as described herein, (ii) the population of stem cells obtained by the method as described herein, or (iii) the composition as described herein, to the subject in a manner effective to confer enhanced hematopoietic activity on a subject.

Also provided is a method of expanding a population of HSC or progenitor cells comprising co-culturing the cells with PDGFRα+ CD51+ mesenspheres in an amount sufficient to can efficiently expand the population of HSC or progenitor cells.

Additional objects of the invention will be apparent from the description which follows.

### BRIEF DESCRIPTION OF THE DRAWINGS

FIG. 1A-1H. Mouse bone marrow (BM) PDGFRα⁺ CD51⁺ cells constitute an enriched population of Nes-GFP⁺ cells. (A) Summary of the mesenchymal, hematopoietic and endothelial cell surface marker antigens screening expressed by stromal Nes-GFP⁺ cells, as detected by FACS analysis. PDGFRα, CD51 and CD105 are expressed by >60% of Nes-GFP⁺ cells. n=3. (B) PDGFRα and CD51 double-positive cells represent a major subpopulation within the Nes-GFP⁺ BM population. (C) BM PDGFRα⁺ CD51⁺ cells directly isolated from the stromal CD45⁻ Ter119⁻ CD31⁻ fraction contain ˜75% of Nes-GFP+ cells. (D) Absolute number of Nes-GFP+ cells expressing PDGFRα and/or CD51 and (E) Number of PDGFRα and CD51 expressing stromal (CD45⁻ Ter119⁻ CD31⁻) cells per mouse femur. Data from n=8 mice. FACS results shown in panels B and C are representative of five independent sorting experiments with similar results. (F) Stromal PDGFRα⁺ CD51⁺ cells isolated from the BM of C57BL/6 mice express high levels of Nestin by real-time PCR gene expression analysis. (G-H) Real-time PCR gene expression analysis of core HSC maintenance and regulation genes in the BM Cxcl12, Vcam1, Angpt1, Opn and Scf within (G) stromal PDGFRα+ CD51+ cells and other indicated subsets isolated from C57BL/6 mice. (H) Within the Nes-GFP+ population, sorted PDGFRα⁺ CD51⁺ cells express the highest levels of HSC maintenance and regulation genes. n=3 independent experiments; *p<0.05; unpaired two-tailed t-test, all error bars indicate SEM.

FIG. 2A-2N. PDGFRα⁺ CD51⁺ BM stromal cells contain the HSC niche activity observed in Nes-GFP⁺ MSCs. (A-J) In vitro characterization of the MSC activity of PDGFRα⁺ CD51⁺ BM cells and other subsets among CD45 Ter119⁻ CD31⁻ stromal cells. (A) Percentage of colony-forming units-fibroblast (CFU-F) in sorted PDGFRα⁺ CD51⁺ cells and other subpopulations. n=3 independent experiments; nd (non-detected). (B) PDGFRα⁺ CD51⁺ cells are able to form self-renewing clonal spheres after 9 days in culture, when plated at clonal densities. n=4 independent experiments. (C, D) When PDGFRα⁺ CD51⁺ cells are isolated from Nes-Gfp mice the clonal spheres formed retain GFP expression for up to ˜1.5 week in culture. (E-J) Multilineage differentiation capacity of PDGFRα⁺ CD51⁺ sphere cultures. Real time PCR gene expression analysis of the differentiation kinetic of PDGFRα⁺ CD51⁺ spheres, showing the upregulation of (E) osteogenic (Gpnmb, Ogn, Sp7), (F) adipogenic (Pparg, Cfd) and (G) chondrogenic (Acan) genes at day 0, 12 and 20 of differentiation; n=3. Fully differentiated phenotypes of PDGFRα⁺ CD51+ spheres shown by (H) Alizarin Red S (osteogenic), (I) lipid vacuole accumulation (adipogenic) and (J) Toluidine Blue (chondrogenic) staining. Single clonal PDGFRα⁺ CD51+ spheres isolated from Nes-Gfp mice were incorporated into (K, L) collagen or (M, N) gelfoam grafts and transplanted under the renal capsule or subcutaneously into recipient mice, respectively. (L, N) Nes-GFP⁺ cells were still detected 8 weeks after implantation, in close contact with host CD45⁺ hematopoietic cells. Cell nuclei were stained with DAPI. White dashed line delineates gelfoam graft borders. (O) Brightfield and (P) fluorescence Nes-GFP⁺ images of secondary PDGFRα⁺ CD51⁺ clonal spheres derived from dissociated gelfoam grafts collected 8 weeks after transplantation. Scale bars: 500 μm (H); 100 μm (D); 50 μm (P, I, J); 20 μm (L, N). *p<0.05; unpaired two-tailed t-test; all error bars indicate SEM.

FIG. 3A-3D. Human fetal BM Nestin⁺ cells express PDGFRα⁺ CD51⁺ cell surface markers. (A) Immunofluorescence staining showing the triple co-localization of a Nestin, PDGFRα and CD51 expressing cell adjacent to bone/cartilage in the human fetal BM. Cell nuclei were stained with DAPI (white). White dashed line delineates the bone/cartilage tissue present in the fetal BM of a 17 gw femur. (B) Representative flow cytometric profiles of freshly isolated stromal (CD45⁻ CD235a⁻ CD31⁻) PDGFRα⁺ CD51⁺ cells in human 19 gw fetal BM. (C) Human stromal PDGFRα⁺ cells express high levels of NESTIN and (D) HSC maintenance genes CXCL12, VCAM1, ANGPT1, OPN and SCF as determined by real-time PCR gene expression analysis. n=3 independent experiments. Scale bar: 20 μm. *p<0.05; unpaired two-tailed t-test; all error bars indicate SEM.

FIG. 4A-4B. Human fetal stromal PDGFRα⁺ CD51⁺CD146^{high} cells express higher levels of HSC maintenance genes than human stromal CD146^{high} cells. (A) Representative FACS profile gating strategy of stromal (CD45⁻ CD235a⁻ CD31⁻), PDGFRα⁺ CD51⁺ (red), PDGFRα⁺ CD51⁺ CD146^{high} (green) and CD146^{high} (blue) populations. CD146^{high} cells contain a small subset (˜30%) of PDGFRα⁺ CD51⁺ expressing cells. (B) Real-time PCR gene expression analysis of core HSC maintenance and regulation genes (CXCL12, VCAM1, ANGPT1, OPN and SCF) in stromal PDGFRα⁺ CD51⁺ (red), PDGFRα⁺ CD51⁺ CD146^{high} (green) and CD146^{high} (blue) cell populations; n=3; *p<0.05; unpaired two-tailed t-test; error bars indicate SEM.

FIG. 5A-5M. HSC niche activity of human fetal BM PDGFRα⁺ CD51⁺ MSCs. (A) The PDGFRα⁺ CD51⁺ human population is significantly enriched for colony forming-units fibroblasts (CFU-Fs) and (B) self-renewing clonal spheres when plated in non-adherent conditions. n=3 independent experiments; nd (non-detected). (C) Example of clonal sphere growth at day 1, 4 and 9. (D-F) Multilineage differentiation capacity of human fetal PDGFRα⁺ CD51⁺ spheres demonstrated by the upregulation of (D) osteoblastic (IBSP, RUNX2, RUNX3), (E) adipogenic (PPARG, SREBF1) and (F) chondrogenic (COL2A1, ACAN, SOX9) lineage differentiation genes during a 21 days period. n=3. (G-I) Fully differentiated phenotypes of human fetal PDGFRα⁺ CD51⁺ spheres shown by (G) Alizarin Red S (osteogenic) staining, (H) lipid vacuole accumulation (adipogenic) and (I) Toluidine Blue (chondrogenic) staining (J-L) Clonally expanded PDGFRα⁺ CD51⁺ human stromal cells are able to establish an ectopic BM microenvironment in a transplantation model. (J) After 8 weeks, hematopoiesis could be detected by the presence of recruited mouse CD45⁺ cells in specific areas across the graft. White dashed line delineates HA/TCP carrier particles. (K-L) Perivascular human self-renewing Nestin⁺ cells were detected in contact with large caliber branching sinusoids containing murine TER119⁺ erythroid cells. Cell nuclei were stained with DAPI. Ft, mesenchymal fibroblastic tissue. (M) Secondary PDGFRα⁺ CD51⁺ clonal spheres derived from dissociated transplanted grafts collected 8 weeks after. Scale bar: 100 μm (G, H, M), 50 μm (C, I), 20 μm (J, L). * p<0.05; unpaired two-tailed t-test, all error bars indicate SEM.

FIG. 6A-6F. PDGFRα⁺ CD51⁺ CD146^{high} mesenspheres show higher HSC expansion potential compared to PDGFRα⁺ CD51⁺ CD146^{high} adherent cells. (CD45⁻ CD235a⁻ CD31⁻) PDGFRα⁺ CD51⁺ were sorted from human fetal bones and grown as mesenspheres under specific conditions (Mendez-Ferrer et al., 2010) or as adherent cells (α-MEM, 10% FBS). (A) Immunophenotypic analysis of human bone marrow mesensphere forming cells and adherent cells. (B-E) Human bone marrow (hBM) CD34+ cells were cultured in serum-free media containing cytokines (Stem Cell Factor, Thrombopoietin and Flt3 Ligand) with human stromal PDGFRα⁺ CD51⁺ CD146^{high} cells previously grown as either mesenspheres or as adherent cells. 9 days after co-culture, human stromal PDGFRα⁺ CD51⁺ CD146^{high} mesenspheres and adherent cells did not show any differences in their ability to support CD45⁺ hematopoietic cells expansion (B). However, mesenspheres yielded a more robust expansion of primitive hematopoietic cell populations (CD45⁺LIN⁻ and CD45⁺LIN⁻CD34⁺) as well as population highly enriched in HSC activity (CD45⁺LIN⁻ CD34⁺CD38⁻) compared to adherent cells (C-E). (F) Expression analysis of HSC maintenance genes in human stromal PDGFRα⁺ CD51⁺ CD146^{high} cells after growing them as either mesenspheres or adherent cells. * p<0.05; **p<0.01; ***p<0.001; unpaired two-tailed t-test, all error bars indicate SEM.

FIG. 7A-7D. PDGFRα⁺ CD51⁺ CD146^{high} human stromal cells expand human HSC enriched population in low cytokine concentration conditions. (A-D) hBM CD34⁺ cells were cultured in serum-free media highly concentrated in cytokines (Stem Cell Factor (100 ng/mL), Thrombopoietin (50 ng/mL) and Flt3 Ligand (100 ng/mL)) with or without PDGFRα⁺ CD51⁺ CD146^{high} mesenspheres. The addition of mesenspheres to human hematopoietic CD34⁺ cells slightly increases the expansion potential of the cytokines on the CD45⁺ (A), CD45⁺LIN⁻ (B), CD45⁺LIN⁻CD34⁺ (C) and CD45⁺LIN⁻CD34⁺ CD38⁻ (D) populations. hBM CD34⁺ cells were then cultured in serum-free media containing low concentration of cytokines (Stem Cell Factor (25 ng/mL), Thrombopoietin (12.5 ng/mL) and Flt3 Ligand (25 ng/mL)) with or without PDGFRα⁺ CD51⁺ CD146^{high} mesenspheres. Under these conditions, the addition of mesenspheres significantly increases the expansion potential of cytokines, to levels similar to the condition with high level of cytokines (A-D). * p<0.05; **p<0.01; ***p<0.001; unpaired two-tailed t-test, all error bars indicate SEM.

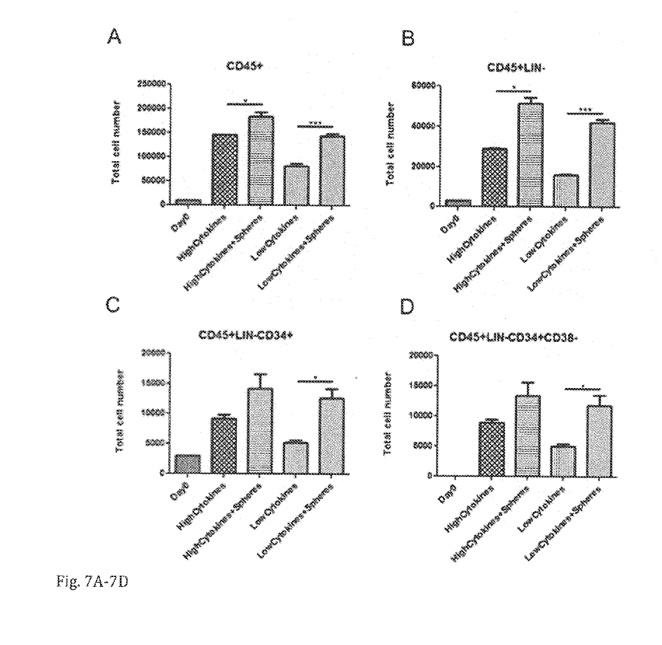

FIG. 8. PDGFRα+CD51+CD146^{high} mesenspheres expand hematopoietic stem and progenitor cells ex vivo. (A) Long-term HSCs were quantified from the input Lin-CD34+ population or after 10 days of co-culture with or without mesenspheres using LTC-IC assay. n=3; *p<0.05; unpaired two-tailed t-test; all error bars indicate SEM. (B) Input CD34+ cells (2×10⁴) or a final culture equivalent to 2×10⁴ CD34+ starting cells cultured with or without mesenspheres were transplanted into NSG mice and human BM engraftment was evaluated 8 weeks post-transplantation. n=10-11 mice per group; *p<0.05; Fisher's exact test; n.s., not significant. (C) Multilineage human hematopoietic engraftment was evaluated by detection of myeloid (CD11b and CD33) and lymphoid (CD19) markers. Representative flow cytometry plots of BM cells from each experimental condition are shown.

### DETAILED DESCRIPTION OF THE INVENTION

This invention provides a method of obtaining a population of stem cells comprising identifying PDGFRα⁺ CD51⁺ cells in a population of cells, and recovering the PDGFRα⁺ CD51⁺ cells so as to obtain the population of stem cells. In an embodiment, the cells are also CD146⁺ and the method comprises identifying CD146⁺ cells. In an embodiment, the cells are CD146^{high}. In a preferred embodiment, the cells are human.

This invention also provides a method of obtaining a population of stem cells comprising identifying PDGFRα⁺ CD51⁺ cells in a population of cells, and separating the PDGFRα⁺ CD51⁺ (αV integrin⁺) cells and recovering the PDGFRα⁺ CD51⁺ cells so as to obtain the population of stem cells. In an embodiment, the cells are also CD146⁺. In an embodiment, the cells are CD146^{high}. In a preferred embodiment, the cells are human.

This invention provides a method of obtaining a population of stem cells comprising identifying PDGFRα⁺ CD51⁺ cells in a heterogeneous population of cells, and recovering the PDGFRα⁺ CD51⁺ cells so as to obtain the population of stem cells. In an embodiment, the method further comprises identifying PDGFRα⁺ CD51⁺ CD146⁺ cells or further comprises identifying PDGFRα⁺ CD51⁺ CD146^{high} cells. In a preferred embodiment, the cells are human.

As used herein, a “heterogeneous” population of cells is a population of cells comprising cells of more than one phenotype, and/or comprising both PDGFRα⁺ CD51⁺ cells and cells which are not PDGFRα⁺ CD51⁺.

In an embodiment of the invention, the population of PDGFRα⁺ CD51⁺ cells is enriched in PDGFRα⁺ CD51⁺ cells above the level of that obtained in a sample obtained from a human subject or occurring naturally. In an embodiment of the invention, the population of PDGFRα⁺ CD51⁺ CD146⁺ cells is enriched in PDGFRα⁺ CD51⁺ CD146⁺ cells above the level of that obtained in a sample obtained from a human subject or occurring naturally.

In an embodiment, recovering the PDGFRα⁺ CD51⁺ cells comprises separating the PDGFRα+CD51+ cells from the heterogeneous population of cells using an antibody, or PDGFRα-binding fragment thereof, directed against PDGFRα and/or using an antibody, or CD51-binding fragment thereof, directed against CD51. In an embodiment, recovering the PDGFRα⁺ CD51⁺ or PDGFRα⁺ CD51⁺ CD146⁺ cells comprises separating the PDGFRα+CD51+ or PDGFRα⁺ CD51⁺ CD146⁺ cells from the heterogeneous population of cells using an antibody, or PDGFRα-binding fragment thereof, directed against PDGFRα and/or using an antibody, or CD51-binding fragment thereof, directed against CD51, and/or using an antibody, or CD146-binding fragment thereof, directed against CD146. In an embodiment, the method comprises using Fluorescence Activated Cell Sorting (FACS) or another immunopurification technique.

In an embodiment the population of cells recovered is further grown in culture or expanded. In an embodiment the population of cells is further grown in the form of non-adherent bodies, for example, spheres.

In an embodiment, red series cells of the sample from which the population is identified are lysed prior to identification or recovery. In an embodiment, the methods further comprise isolating CD45− cells prior to identifying the PDGFRα⁺ CD51⁺ CD146⁺ cells or PDGFRα⁺ CD51⁺ cells.

In an embodiment, the heterogeneous population of cells is a population of bone marrow cells. In an embodiment, the heterogeneous population of cells is a heterogeneous population of stem cells. In an embodiment, the stem cells are human stem cells. In an embodiment, the stem cells are mesenchymal stem cells. In a preferred embodiment, the population of stem cells obtained is a population of human mesenchymal stem cells.

In an embodiment, the population of stem cells is 5% or greater, 10% or greater, 15% or greater, 20% or greater, 25% or greater, 30% or greater, 35% or greater, 40% or greater, or 45% or greater PDGFRα⁺ CD51⁺. In an embodiment, the population of stem cells is 50% or greater PDGFRα⁺ CD51⁺. In an embodiment, the population of stem cells is 75% or greater PDGFRα⁺ CD51⁺. In an embodiment, the population of stem cells is 90% or greater PDGFRα⁺ CD51⁺. In an embodiment, the population of stem cells is 5% or greater, 10% or greater, 15% or greater, 20% or greater, 25% or greater, 30% or greater, 35% or greater, 40% or greater, or 45% or greater PDGFRα⁺ CD51⁺ CD146⁺. In an embodiment, the population of stem cells is 5% or greater, 10% or greater, 15% or greater, 20% or greater, 25% or greater, 30% or greater, 35% or greater, 40% or greater, or 45% or greater PDGFRα⁺ CD51⁺ CD146^{high}.

In an embodiment, the population of cells or the population of stem cells are CD45⁻ Ter119⁻ CD31⁻. In an embodiment, the population of stem cells are nestin positive (nestin⁺). In an embodiment, the population of stem cells are one or more of CD45, CD235a⁻, and CD31⁻. In an embodiment, the population of stem cells are CD45⁻ CD235a⁻ CD31⁻ and are human.

In an embodiment, the methods further comprise recovering CD105⁺ cells from the PDGFRα⁺ CD51⁺ or the PDGFRα⁺ CD51⁺ CD146⁺ or the PDGFRα⁺ CD51⁺ CD146^{high} population of stem cells.

In an embodiment, the methods further comprise expanding the population of PDGFRα⁺ CD51⁺ or PDGFRα⁺ CD51⁺ CD146⁺ or PDGFRα⁺ CD51⁺ CD146^{high} stem cells in culture. In an embodiment, the methods further comprise recovering the expanded population of stem cells. In a preferred embodiment, the cells are expanded as as non-adherent clonal mesenspheres.

In an embodiment, the PDGFRα⁺ CD51⁺ cells are obtained by a technique comprising identifying the PDGFRα⁺ cells using an antibody directed against PDGFRα and then identifying the CD51⁺ cells of the PDGFRα⁺ cells using an antibody directed against CD51. In an embodiment, the PDGFRα⁺ CD51⁺ cells are obtained by a technique comprising identifying the CD51⁺ cells using an antibody directed against CD51 and then identifying the PDGFRα⁺ cells of the CD51⁺ cells using an antibody directed against PDGFRα. In an embodiment, one or both of the antibodies are attached to an affinity column. In an embodiment, the PDGFRα⁺ CD51⁺ cells are obtained by a technique comprising sequential immunopurification of the PDGFRα⁺ cells then the CD51⁺ cells subpopulation or sequential immunopurification of the CD51⁺ cells then the PDGFRα⁺ cells subpopulation. In an embodiment, the PDGFRα⁺ CD51⁺ cells are obtained by a technique comprising immunopurification of the PDGFRα⁺ CD51⁺ cells with a PDGFRα, CD51 bispecific antibody. In an embodiment wherein PDGFRα⁺ CD51⁺ CD146+ or PDGFRα⁺ CD51⁺ CD146⁺ cells are to be obtained, the method further comprises identifying such cells using an antibody, or CD146-binding fragment thereof, directed against CD146. Thus, the method may comprise sequential purification using a CD146 antibody, a PDGFRα⁺ antibody and a CD51⁺ antibody in any order.

Also provided is an isolated population of PDGFRα⁺ CD51⁺ mesenchymal stem cells, wherein the population is enriched in PDGFRα⁺ CD51⁺ cells above the naturally occurring level of the cells in a naturally occurring population of cells. Also provided is an isolated population of PDGFRα⁺ CD51⁺ CD146⁺ mesenchymal stem cells, wherein the population is enriched in PDGFRα⁺ CD51⁺ CD146⁺ cells above the naturally occurring level of the cells in a naturally occurring population of cells. Also provided is an isolated population of PDGFRα⁺ CD51⁺ CD146^{high} mesenchymal stem cells, wherein the population is enriched in PDGFRα⁺ CD51⁺ CD146^{h1} cells above the naturally occurring level of the cells in a naturally occurring population of cells. In a preferred embodiment, the cells are human.

In an embodiment, the population is 5% or greater, 10% or greater, 15% or greater, 20% or greater, 25% or greater, 30% or greater, 35% or greater, 40% or greater, or 45% or greater, or 50% or greater PDGFRα⁺ CD51⁺. In an embodiment, the population is 50% or greater PDGFRα⁺ CD51⁺ cells. In an embodiment, the population is 75% or greater PDGFRα⁺ CD51⁺ cells. In an embodiment, the population is 90% or greater PDGFRα⁺ CD51⁺ cells. In an embodiment, the population is 5% or greater, 10% or greater, 15% or greater, 20% or greater, 25% or greater, 30% or greater, 35% or greater, 40% or greater, or 45% or greater, or 50% or greater PDGFRα⁺ CD51⁺ CD146⁺ cells. In an embodiment, the population is 75% or greater PDGFRα⁺ CD51⁺ cells. In an embodiment, the population is 90% or greater PDGFRα⁺ CD51⁺ CD146⁺ cells. In an embodiment, the population is 5% or greater, 10% or greater, 15% or greater, 20% or greater, 25% or greater, 30% or greater, 35% or greater, 40% or greater, or 45% or greater, or 50% or greater PDGFRα⁺ CD51⁺ CD146^{high} cells. In an embodiment, the population is 75% or greater PDGFRα⁺ CD51⁺ cells. In an embodiment, the population is 90% or greater PDGFRα⁺ CD51⁺ CD146^{high} cells.

In an embodiment, the isolated population has CFU-F activity. In an embodiment, the PDGFRα⁺ CD51⁺ cells are multipotent. In an embodiment, the PDGFRα⁺ CD51⁺ cells are osteogenic, adipogenic and/or chondrogenic or capable of osteogenic, adipogenic and/or chondrogenic differentiation. In an embodiment, the PDGFRα⁺ CD51⁺ cells are also CD146⁺. In an embodiment, the PDGFRα⁺ CD51⁺ cells are also CD146^{high}. In an embodiment, the PDGFRα⁺ CD51⁺ cells are also CD105⁺.

Also provided is a method comprising co-culturing a population of cells comprising stem cells, with any of the above described PDGFRα⁺ CD51⁺ cells or populations of such cells, so as to produce an expanded population of stem cells. In an embodiment, the stem cells are hematopoietic stem cells. In an embodiment, the method further comprises recovering the expanded population of stem cells. In an embodiment, the population comprises mesenchymal stem cells, preferably a population of bone marrow cells. In an embodiment, the population comprises stem cells is a population of human cells. In an embodiment, the cells are grown as non-adherent clonal spheres. In a preferred embodiment, the population of cells comprising stem cells are co-cultured with PDGFRα⁺ CD51⁺ CD146⁺ cells, preferably PDGFRα⁺ CD51⁺ CD146^{high} cells.

Also provided is a composition comprising any of the above-described PDGFRα⁺ CD51⁺ cells, or population of such cells, and a carrier. In an embodiment, the carrier is a pharmaceutically acceptable carrier. In an embodiment, the composition is a pharmaceutical composition. Also provided is a composition comprising any of the above-described PDGFRα⁺ CD51⁺ CD146⁺ cells, or population of such cells, and a carrier. In an embodiment, the carrier is a pharmaceutically acceptable carrier. In an embodiment, the composition is a pharmaceutical composition. Also provided is a composition comprising any of the above-described PDGFRα⁺ CD51⁺ CD146^{high} cells, or population of such cells, and a carrier. In an embodiment, the carrier is a pharmaceutically acceptable carrier. In an embodiment, the composition is a pharmaceutical composition.

Also provided is a method comprising administering an amount of any of the described populations of stem cells, or the described compositions, to a subject, in an amount effective to confer stem cell activity on a subject. In an embodiment, the amount is effective to confer hematopoietic activity.

Also provided is a method of enhancing hematopoietic acitivty in a subject comprising administering an amount of (i) the population of stem cells as described herein, (ii) the population of stem cells obtained by the method as described herein, or (iii) the composition as described herein, to the subject in a manner effective to confer enhanced hematopoietic acitivty on a subject. In an embodiment, human PDGFRα+ CD51+ mesenspheres are administered.

Also provided is a method of expanding a population of HSC or progenitor cells comprising co-culturing the cells with PDGFRα+ CD51+ mesenspheres in an amount sufficient to can efficiently expand expand the population of HSC or progenitor cells.

In an embodiment, the HSC or progenitor cells are CD34+ cells. In an embodiment, the HSC or progenitor cells are obtained from bone marrow.

As used herein, the term “antibody” refers to an intact antibody, i.e. with complete Fc and Fv regions. “Fragment” refers to any portion of an antibody, or portions of an antibody linked together, such as a single-chain Fv (scFv), which is less than the whole antibody but which is an antigen-binding portion and which competes with the intact antibody of which it is a fragment for specific binding. As such a fragment can be prepared, for example, by cleaving an intact antibody or by recombinant means. See generally, Fundamental Immunology, Ch. 7 (Paul, W., ed., 2nd ed. Raven Press, N.Y. (1989), hereby incorporated by reference in its entirety). Antigen-binding fragments may be produced by recombinant DNA techniques or by enzymatic or chemical cleavage of intact antibodies or by molecular biology techniques. In some embodiments, a fragment is an Fab, Fab′, F(ab′)₂, F_{d}, Fᵥ, complementarity determining region (CDR) fragment, single-chain antibody (scFv), (a variable domain light chain (V_{L}) and a variable domain heavy chain (V_{H}) linked via a peptide linker. In an embodiment the linker of the scFv is 10-25 amino acids in length. In an embodiment the peptide linker comprises glycine, serine and/or threonine residues. For example, see Bird et al., Science, 242: 423-426 (1988) and Huston et al., Proc. Natl. Acad. Sci. USA, 85:5879-5883 (1988) each of which are hereby incorporated by reference in their entirety), or a polypeptide that contains at least a portion of an antibody that is sufficient to confer human βV-tubulin-specific antigen binding on the polypeptide, including a diabody. From N-terminus to C-terminus, both the mature light and heavy chain variable domains comprise the regions FR1, CDR1, FR2, CDR2, FR3, CDR3 and FR4. The assignment of amino acids to each domain is in accordance with the definitions of Kabat, Sequences of Proteins of Immunological Interest (National Institutes of Health, Bethesda, Md. (1987 and 1991)), Chothia & Lesk, J. Mol. Biol. 196:901-917 (1987), or Chothia et al., Nature 342:878-883 (1989), each of which are hereby incorporated by reference in their entirety). As used herein, the term “polypeptide” encompasses native or artificial proteins, protein fragments and polypeptide analogs of a protein sequence. A polypeptide may be monomeric or polymeric. As used herein, an F_{d} fragment means an antibody fragment that consists of the V_{H} and CH1 domains; an Fᵥ fragment consists of the V₁ and V_{H} domains of a single arm of an antibody; and a dAb fragment (Ward et al., Nature 341:544-546 (1989) hereby incorporated by reference in its entirety) consists of a V_{H} domain.

Depending on the amino acid sequences of the constant domains of their heavy chains, antibodies (immunoglobulins) can be assigned to different classes. The antibody or fragment can be, e.g., any of an IgG, IgD, IgE, IgA or IgM antibody or fragment thereof, respectively. In an embodiment the antibody is an immunoglobulin G. In an embodiment the antibody fragment is a fragment of an immunoglobulin G. In an embodiment the antibody is an IgG1, IgG2, IgG2a, IgG2b, IgG3 or IgG4. In an embodiment the antibody comprises sequences from a human IgG1, human IgG2, human IgG2a, human IgG2b, human IgG3 or human IgG4. A combination of any of these antibodies subtypes can also be used. One consideration in selecting the type of antibody to be used is the desired serum half-life of the antibody. For example, an IgG generally has a serum half-life of 23 days, IgA 6 days, IgM 5 days, IgD 3 days, and IgE 2 days. (Abbas A K, Lichtman A H, Pober J S. Cellular and Molecular Immunology, 4th edition, W.B. Saunders Co., Philadelphia, 2000, hereby incorporated by reference in its entirety).

In an embodiment, the compositions of the invention, for example comprising the above-described cells or populations of cells, comprise a pharmaceutically acceptable carrier. Examples of pharmaceutically acceptable carriers include, but are not limited to, phosphate buffered saline solution, osmotically balanced sterile water, and other carriers compatible with stem cell viability and administration to a mammalian subject.

In a preferred embodiment of the inventions described herein, the subject is a human.

All combinations of the various elements described herein are within the scope of the invention unless otherwise indicated herein or otherwise clearly contradicted by context.

This invention will be better understood from the Experimental Details, which follow. However, one skilled in the art will readily appreciate that the specific methods and results discussed are merely illustrative of the invention as described more fully in the claims that follow thereafter.

### EXPERIMENTAL DETAILS

### Introduction

Herein, cell surface MSC receptors have been evaluated to identify a stromal population equivalent to nestin⁺ cells. The results show that the combination of PDGFRα and CD51 (and CD146^{high} in humans) identifies a large subset of nestin⁺ cells that is highly enriched in MSC and HSC niche activities. Further, it is shown that PDGFRα⁺ CD51⁺ stromal cells isolated from human BM can also form self-renewing clonal mesenspheres capable of transferring hematopoietic niche activity in vivo and for expanding hematopoietic stem cell in vitro.

### Example 1

### Materials and Methods

Mouse strains: All murine experiments were performed using adult 8-12 weeks old animals. All mice were housed in specific pathogen-free facilities at the Albert Einstein College of Medicine (AECOM) animal facility and all the experimental procedures approved by the Animal Care and Use Committee of the AECOM. C57BL/6 mice were purchased from National Cancer Institute (Frederick Cancer Research Center, Frederick, Md.). Nes-Gfp transgenic mice (Mignone et al., 2004) were at AECOM. For the human fetal cells in vivo transplantation, NOD-scid Il2rg^{-/-} (NSG) immunocompromised mice were used and bred at AECOM.

Cell isolation: Bone marrow primary cells were isolated as previously described (Mendez-Ferrer et al., 2010) with minor modifications. Briefly, femora, tibia and humeri bone marrow was gently flushed in L-15 FACS buffer (Mendez-Ferrer et al., 2010) and after erythrocyte lysis, digested with 1 mg/ml collagenase IV (Sigma) in HBSS (Gibco) with 10% fetal bovine serum (FBS) (StemCell Technologies), 30 min at 37° C. For flow cytometry sorting, cells were enriched by immunomagnetic depletion using anti-CD45 magnetic beads (Milteyi Biotec), following the manufacturer's recommendations. Cells were sorted on a FACSAria (BD) to >95% purity. Human fetal bone marrow samples, between 13-20 gw, were obtained from the AECOM Human Fetal Tissue Repository by protocols approved by the AECOM Institutional Review Board.

Flow Cytometry: Fluorochrome-conjugated or biotinylated mAbs specific to mouse CD45 (clone 30-F11), Ter119 (clone Ter-119), PDGFRα(clone APA5), CD51 (clone RMV-7), CD44 (clone IM7), CD130 (clone KGP130), c-Kit (clone 2B8), CD135 (clone A2F10), CD90 (clone 53-2.1), CD34 (clone RAM34), CD166 (clone eBioALC48), Sca1 (clone D7), CD41 (clone MWReg30), CD133 (clone 13A4), CD11b (clone M1/70) and corresponding isotype controls were purchased from Ebioscience. P75 (clone 2E3), CD10 (clone EPR2997) and Nrp1 (clone EPR3113) were purchased from Abcam. CD31 (clone MEC13.3), CD105 (clone MJ7/18) and CD48 (clone HM48-1) were from Biolegend while CD29 (clone KMI6) and CD146 (clone ME-9F1) were from BD Biosciences. Ng2 rabbit polyclonal was obtained from Millipore. Secondary antibodies Alexa Fluor® 633 Goat Anti-Rabbit IgG and Alexa Fluor® 633 Goat Anti-Rat IgG were from Molecular Probes. Fluorochrome-conjugated mAbs specific to human CD45 (clone 2D1), CD235a (clone HIR2) and CD31 (clone WM59) were from Ebioscience. PDGFRα(clone αR1) and CD146 (clone PIH12) were purchased from BD Bioscience and finally CD51 (clone NKI-M9) from Biolegend. Nes-GFP positive staining was gated in reference to cells from wild-type mice without the GFP transgene and positive specific antibodies labeling were gated in reference to corresponding isotype control or fluorescence-minus-one (FMO) corresponding sample. Multiparameter analyses of stained cell suspensions were performed on an LSRII (BD) and analyzed with FlowJo software (Tree Star). DAPI—single cells were evaluated for all the analyses.

Cell culture and differentiation: For clonal sphere formation, cells were plated at clonal density (<500 cells/cm²) or by single cell sorting into ultra-low adherent plates as previously described (Mendez-Ferrer et al., 2010). Cells were kept at 37° C. with 5% CO₂ in a water-jacketed incubator and left untouched for one week to prevent cell aggregation. One-half medium changes were performed weekly. All spheres in a given well were counted at day 9 and results expressed as a percentage of plated cells.

For osteogenic, adipogenic and chondrogenic differentiation, mouse or human PDGFRα⁺ CD51⁺ cells were treated with StemXVivo Osteogenic, Adipogenic or Chondrogenic mouse or human specific differentiation media, according to manufacturer's instructions (R&D Systems). All cultures were maintained with 5% CO₂ in a water-jacketed incubator at 37° C. At specific time points, cells were collected for RNA or cytochemistry analysis. Osteogenic differentiation indicated by mineralization of extracellular matrix and calcium deposits was revealed by Alizarin Red S staining. Cells were fixed with 4% paraformaldehyde (PFA) for 30 min. After rinsing in distilled water, cells were stained with 40 mM Alizarin Red S (Sigma-Aldrich) solution at pH 4.2, rinsed in distilled water, and washed in Tris-buffered saline for 15 min to remove nonspecific stain. Adipocytes were identified by the typical production of lipid droplets. Chondrocytes were revealed by Toluidine Blue staining, which detects the synthesis of glycosaminoglycan. Cells were fixed with 4% PFA for 60 min, embedded in paraffin and sectioned. Sections were incubated with 0.5% Toluidine Blue (Sigma-Aldrich) in distilled water for 15 min. To remove nonspecific stain, sections were rinsed 3 times with running water (5 min each).

CFU-F assay: Mouse 1-3×10³ sorted cells were seeded per well in a 12-well adherent tissue culture plate using phenol-red free α-MEM (Gibco) supplemented with 20% FBS (Hyclone), 10% MesenCult stimulatory supplement (StemCell Technologies) and 0.5% penicillin-streptomycin. One-half of the media was replaced after 7 days and at day 14 cells were stained with Giemsa staining solution (EMD Chemicals). Human fetal bone marrow cells were plated at 0.5-1×10³ cells/well into 12 well adherent tissue culture plates using phenol-red free α-MEM (Gibco) with 20% FBS (StemCell Technologies) and 0.5% penicillin-streptomycin. One-half of the media was replaced after 5 days and at day 10 cells were stained and adherent colonies counted.

RNA isolation and quantitative real-time PCR: Sorted or cultured cells were collected in lysis buffer and RNA isolation was performed using the Dynabeads® mRNA DIRECT™ Micro Kit (Invitrogen). Reverse transcription was performed using the RNA to cDNA EcoDry™ Premix system (Clontech), following the manufacturer's recommendations. Quantitative real-time PCR was performed as previously described (Mendez-Ferrer et al., 2010). Human and mouse primer sequences are included in Table 1.

Immunofluorescence staining: Human staining's were performed on whole mount non-fixed and non-decalcified bones. Hydroxyapatite/tricalcium phosphate (HA/TCP) grafts were fixed with 4% PFA during 2 h at 4° C., partially decalcified with 0.25 M EDTA for 3-5 days and cryoprotected with 15-30% sucrose. Grafts were then processed as described (Kawamoto, 2003) and immunostained using standard technique. Collagen and gelfoam grafts were also processed as above described without the decalcification step and using Superfrost/Plus slides (Fisher Scientific). The following antibodies were used as primary: Alexa Fluor® 488 anti-GFP (1:200, Molecular Probes); anti-mouse CD45-Pe (1:200; clone 30-F11, Ebioscience); anti-mouse Ter119-Pe and biotinylated (1:200; clone Ter119, Ebioscience); anti-human Nestin (1:200; clone 196908, R&D systems); anti-human PDGFRα(1:200, clone C-20, Santa Cruz Biotechnology); anti-human CD51-FITC (1:200, clone NKI-M9, Biolegend) and anti-human biotinylated CD146 (1:200, clone 541-10B2, Milteyi Biotec). The secondary antibodies used were Alexa Fluor® 633 goat anti-mouse IgG, Alexa Fluor® 568 goat anti-rabbit IgG and Alexa Fluor® 488 goat anti-mouse IgG all at 1:500 (Molecular probes). APC-streptavidin solution (Jackson Laboratories) was also used for biotinylated antibodies. For nuclear staining, samples were treated with DAPI (Sigma). Images were captured using an Axio Examiner D1 confocal microscope (Zeiss) and images processed using the SlideBook software (Intelligent Imaging Innovations).

In vivo transplantations: For renal capsule collagen graft, five thousand freshly sorted cells, or single spheres were gently re-suspended in 15 μl of a collagen (BD Biosciences) mixed with 2% 1N NaOH and 10% 10×PBS. The cells/collagen mix were then gently deposited into a 6-well plate and incubated at 37° C. for 30 min to allow the collagen to solidify. Collagen grafts were then transplanted under the renal capsule of 8-12 week old anaesthetized mice. After 8 weeks, kidneys/grafts were collected and processed for immunofluorescence analysis.

For subcutaneous gelfoam graft, transplantations were performed as previously described (Bianco et al., 2006) with minor alterations. Five thousand freshly sorted cells or single spheres were gently re-suspended in 50 μl of spheres media. Five mm³ cubes of sterile collagen sponges (Gelfoam, Pfizer) were hydrated into spheres media and then squeezed to remove air bubbles and allow the sponge to regain its size. Just before transplantation, sponges were blotted between two pieces of sterile filter paper and placed in contact with the cells mixture at 37° C. for 90 min. As the sponges expanded, they incorporate the cells. Gelfoam grafts were then implanted subcutaneously under the dorsal skin of 8-12 week-old anaesthetized recipient animals. After 8 weeks gelfoam grafts were collected and processed for immunofluorescence analysis.

For subcutaneous HA/TCP graft, transplantation of human fetal cells was performed as described (Kuznetsov et al., 1997) with minor modifications. 5×10⁵ cells derived from a clonally expanded sphere or 5×10⁵ non-clonal expanded cells re-suspended into sphere media were allowed to attach the HA/TCP powder (Ceraform, Teknimed S A) by slow rotation at 37° C. After 60 min cells mixture was spun and media replaced by collagen (BD Biosciences) mixed with 2% 1N NaOH and 10% 10×PBS. Grafts were incubated for another 30 min at 37° C. and transplanted s.c. into 8-12 week old female NSG anaesthetized recipient mice. After 8 weeks HA/TCP grafts were collected and processed for immunofluorescence and histological analysis as described (Kuznetsov et al., 1997).

For co-culture experiments: (CD45⁻ CD235a⁻ CD31⁻) PDGFRα⁺ CD51⁺ were sorted from human fetal bones and grown as mesenspheres under specific conditions (Mendez-Ferrer et al., 2010) or as adherent cells (α-MEM, 10% FBS). Human fetal bone marrow cells were incubated with magnetic beads coupled to anti-human CD34 antibobies and human bone marrow (hBM) CD34+ cells were positively selected after eluting them from a magnetic column. CD34+ cells were cultured in serum-free media containing cytokines (Stem Cell Factor, Thrombopoietin and Flt3 Ligand) for 9 days with human stromal PDGFRα⁺ CD51⁺ CD146^{high} cells previously grown as either mesenspheres or as adherent cells. hBM CD34⁺ cells were cultured in serum-free media highly concentrated in cytokines (Stem Cell Factor (100 ng/mL), Thrombopoietin (50 ng/mL) and Flt3 Ligand (100 ng/mL) with or without PDGFRα⁺ CD51⁺ CD146^{high} mesenspheres. hBM CD34⁺ cells were then cultured in serum-free media containing low concentration of cytokines (Stem Cell Factor (25 ng/mL), Thrombopoietin (12.5 ng/mL) and Flt3 Ligand (25 ng/mL)) with or without PDGFRα⁺ CD51⁺ CD146^{high} mesenspheres.

TABLE 1
Primers used (SEQ ID NOS: 1-56,
top to bottom, respectively).

| | | Sequence 5′-3′ |
|---|---|---|
| Human | | |
| primers | | |
| GAPDH | s | TCTGCTCCTCCTGTTCGACA |
| | as | AAAAGCAGCCCTGGTGACC |
| CXCL12 | s | TGGGCTCCTACTGTAAGGGTT |
| | as | TTGACCCGAAGCTAAAGTGG |
| VCAM1 | s | GTCTCCAATCTGAGCAGCAA |
| | as | TGAGGATGGAAGATTCTGGA |
| ANGPT1 | s | GCCATCTCCGACTTCATGTT |
| | as | CTGCAGAGAGATGCTCCACA |
| OPN | s | AGATGGGTCAGGGTTTAGCC |
| | as | CATCACCTGTGCCATACCAG |
| SCF | s | AATCCTCTCGTCAAAACTGAAGG |
| | as | CCATCTCGCTTATCCAACAATGA |
| NESTIN | s | GGGAGTTCTCAGCCTCCAG |
| | as | GGAGAAACAGGGCCTACAGA |
| IBSP | s | TGAAGTCTCCTCTTCTTCCTCCT |
| | as | AAACGATTTCCAGTTCAGGG |
| RUNX2 | s | ATACTGGGATGAGGAATGCG |
| | as | ACAGTAGATGGACCTCGGGA |
| RUNX3 | s | GTCTGGTCCTCCAGCTTCTG |
| | as | CTGTGTTCACCAACCCCAC |
| PPARG | s | GAGAGATCCACGGAGCTGAT |
| | as | AGGCCATTTTGTCAAACGAG |
| SREBF1 | s | GTTGGCCCTACCCCTCC |
| | as | CTTCAGCGAGGCGGCTT |
| COL2A1 | s | TTTCTGTCCCTTTGGTCCTG |
| | as | GTGAGCCATGATTCGCCTC |
| ACAN | s | GCGAGTTGTCATGGTCTGAA |
| | as | TTCTTGGAGAAGGGAGTCCA |
| SOX9 | s | GTAATCCGGGTGGTCCTTCT |
| | as | GACGCTGGGCAAGCTCT |
| Mouse | | |
| primers | | |
| Gapdh | s | TGTGTCCGTCGTGGATCTGA |
| | as | CCTGCTTCACCACCTTCTTGA |
| Cxcl12 | s | CGCCAAGGTCGTCGCCG |
| | as | TTGGCTCTGGCGATGTGGC |
| Vcam1 | s | GACCTGTTCCAGCGAGGGTCTA |
| | as | CTTCCATCCTCATAGCAATTAAGGTG |
| Angpt1 | s | CTCGTCAGACATTCATCATCCAG |
| | as | CACCTTCTTTAGTGCAAAGGCT |
| Opn | s | TCCCTCGATGTCATCCCTGTTG |
| | as | GGCACTCTCCTGGCTCTCTTTG |
| Scf | s | CCCTGAAGACTCGGGCCTA |
| | as | CAATTACAAGCGAAATGAGAGCC |
| Nestin | s | GCTGGAACAGAGATTGGAAGG |
| | as | CCAGGATCTGAGCGATCTGAC |
| Gpnmb | s | CCCCAAGCACAGACTTTTGAG |
| | as | GCTTTCTGCATCTCCAGCCT |
| Ogn | s | ACCATAACGACCTGGAATCTGT |
| | as | AACGAGTGTCATTAGCCTTGC |
| Sp7 | s | ATGGCGTCCTCTCTGCTTGA |
| | as | GAAGGGTGGGTAGTCATTTG |
| Pparg | s | ACCACTCGCATTCCTTTGAC |
| | as | TGGGTCAGCTCTTGTGAATG |
| Cfd | s | TGCATCAACTCAGAGTGTCAATCA |
| | as | TGCGCAGATTGCAGGTTGT |
| Acan | s | CACGCTACACCCTGGACTTTG |
| | as | CCATCTCCTCAGCGAAGCAGT |

### Results

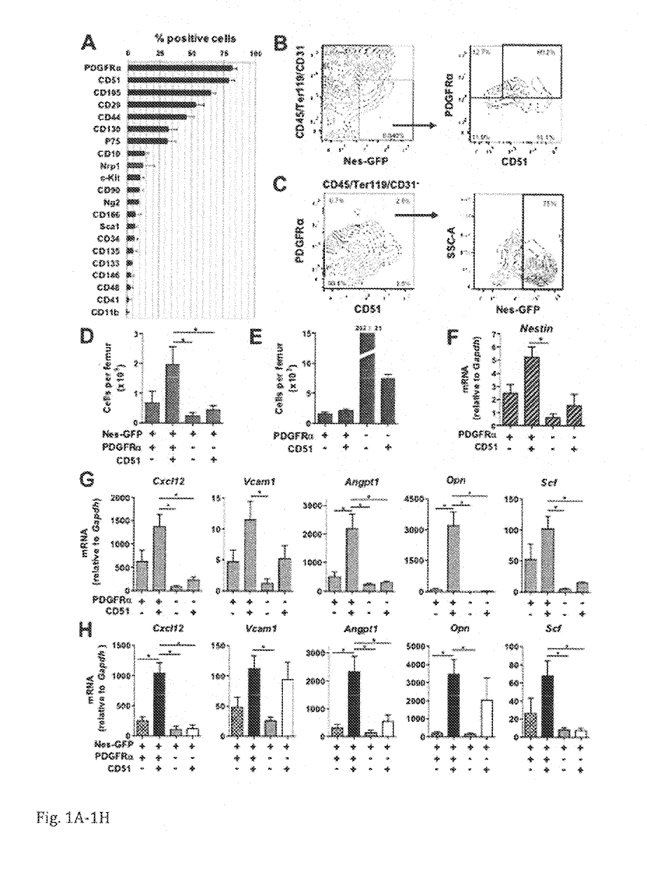

PDGFRα and CD51 label most Nes-GFP+ cells: To identify the cell surface marker(s) equivalent of Nestin⁺ cells, microarray data were used (Mendez-Ferrer et al., 2010) and previously published MSC markers. Among non-hematopoietic (CD45⁻Ter119⁻) and non-endothelial (CD31) Nes-GFP⁺ cells dissociated with collagenase type IV, platelet-derived growth factor receptor alpha (PDGFRα) and αV integrin (CD51) were highly and uniformly expressed by BM Nestin⁺ cells (82±3% and 79±4%, respectively; FIG. 1A). Another putative MSC marker, endoglin (CD105), was also expressed by 65±3% of Nestin⁺ cells. Other conventional mesenchymal lineage markers were heterogeneously expressed (CD29, CD44, CD130, P75) or restricted to a small subset (<15%) of Nestin⁺ cells (CD10, Nrp1, CD166, CD133). Ng2 (Ozerdem et al., 2001) and CD146 (Li et al., 2003; Sacchetti et al., 2007), two known perivascular markers, along with the putative MSC markers Sca1 (Meirelles Lda and Nardi, 2003; Morikawa et al., 2009) and CD90 (Pittenger et al., 1999), were also expressed in a very small fraction of BM Nestin⁺ cells (<10%). As expected, various hematopoietic markers (c-Kit, CD135, CD48, CD41, CD11b and CD34) were absent or expressed <10% of Nestin+ cells (FIG. 1A).

Next, the analysis of the combination of the three most highly expressed markers (CD105, PDGFRα and CD51) showed that only PDGFRα and CD51 double-positive cells were capable of faithfully identify the Nes-GFP population. PDGFRα and CD51 double-positive cells comprised a major subset of the Nes-GFP⁺ population (˜60%; FIGS. 1B and D). By gating first on PDGFRα⁺ CD51⁺ cells, they represented a rare fraction (˜2%) of the CD45⁻ Ter119⁻ CD31⁻ stromal population, but were highly enriched in Nes-GFP⁺ cells (˜75%; FIGS. 1C and E). Endogenous Nestin expression, as seen by real-time PCR, was also enriched in PDGFRα⁺ CD51+ cells, compared to single-positive or negative stromal cells (FIG. 1F).

Stromal PDGFRα⁺ CD51⁺ cells express high levels of HSC maintenance and regulatory genes: Nestin+ cells express high levels of HSC maintenance genes such as the chemokine Cxcl12, vascular cell adhesion molecule-1 (Vcam1), angiopoietin-1 (Angpt1), stem cell factor (Scf), and osteopontin (Opn) (Mendez-Ferrer et al., 2010). CD105 PDGFRα CD51 double- and single-positive subsets were sorted among stromal cells (CD45⁻ Ter119⁻CD31⁻) to evaluate their niche properties (FIG. 1C). It was found that PDGFRα and CD51 double-positive cells consistently enriched for the highest levels of HSC regulatory genes (FIG. 1G). Moreover, within the Nes-GFP⁺ fraction, the PDGFRα⁺ CD51⁺ subset also expressed the highest levels of these factors (FIG. 1H). To confirm this finding, the expression levels between PDGFRα⁺ CD51⁺ cells were also compared with the small fraction of Nes-GFP⁺ cells that do not express PDGFRα and CD51. Approximately 1.3% of these cells were Nes-GFP⁺ and expressed significantly lower levels of HSC maintenance factors compared to the entire PDGFRα⁺ CD51⁺ population (of which ˜75% are Nes-GFP⁺). Furthermore, the gene expression analysis showed that within the PDGFRα⁺ CD51⁺ population, a small fraction of Nes-GFP⁻ cells (˜25%) also expresses considerable levels HSC-niche genes, in particular Opn and Scf. These results show that PDGFRα⁺ CD51⁺ stromal cells express the key HSC niche genes contained in Nestin⁺ cells and suggest that this population may represent a suitable alternative to prospectively isolate niche cells.

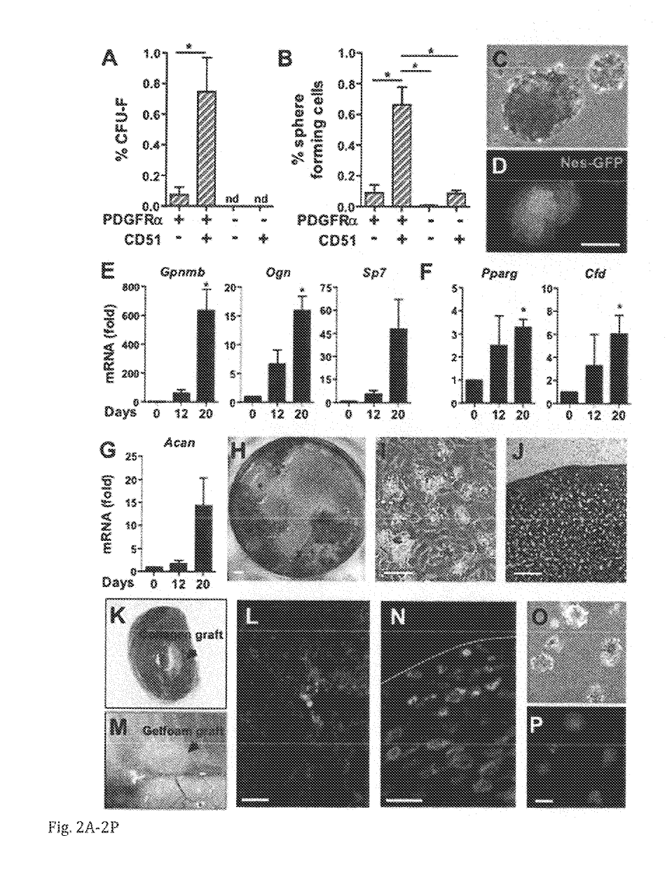

PDGFRα⁺ CD51⁺ BM stromal cells recapitulate the MSC identity of Nestin⁺ cells: Nes-GFP⁺ cells comprise all the MSC activity in BM, as determined by the exclusive ability to form CFU-F and mesenspheres that can self-renew in vivo (Mendez-Ferrer et al., 2010). Since both MSC and HSC niche activities are very rare in BM, and likely found in a subset of Nes-GFP⁺ cells, it remains possible that the two activities are not conferred by the same cell. Having found that niche activity is enriched in PDGFRα⁺ CD51⁺ cells which comprised 60% of Nes-GFP⁺ cells, it was next tested whether MSC activity co-segregates with the niche function. CFU-F assays of sorted double- and single-positive fractions revealed that mesenchymal progenitor activity was only present in the stromal PDGFRα⁺ CD51⁺ fraction (FIG. 2A), as seen for Nestin⁺ cells (Mendez-Ferrer et al., 2010). In addition, PDGFRα⁺ CD51⁺ cells, in contrast to other stromal subpopulations plated at clonal densities (<500 cells/cm²) or by single-cell FACS sorting deposition, were able to form efficiently non-adherent primary spheres (FIG. 2B). When dissociated, these spheres could be passaged, forming secondary spheres, demonstrating the in vitro self-renewal capacity of PDGFRα⁺ CD51⁺ cells. By contrast, the rare and small spheres (<40 μm in diameter) forming from PDGFRα⁺ CD51⁻ and PDGFRα⁻ CD51⁺ subpopulations (FIG. 2B) did not have the capacity to form secondary spheres in culture. When PDGFRα⁺ CD51+ cells were isolated from Nes-Gfp mice the majority of the clonal spheres with sizes typically ranging from 40 to 130 μm in diameter, retained Nes-GFP expression until ˜1.5 week in culture (FIGS. 2C and D). Using conventional adherent MSC culture conditions (Phinney et al., 1999; Pittenger et al., 1999), sorted PDGFRα⁺ CD51⁺ cells rapidly downregulated HSC-maintenance genes expression along with Nes-GFP (data not shown). Clonally expanded PDGFRα⁺ CD51⁺ spheres plated into in vitro mesenchymal lineage differentiation conditions exhibited robust tri-lineage potential, with upregulation of osteoblastic (FIG. 2E), adipocytic (FIG. 2F) and chondrocytic (FIG. 2G) differentiation genes during a 12-20 days period. Multilineage differentiation was confirmed by morphological and histochemical characterization of mature mesenchymal lineage phenotypes after >30 days in culture (FIG. 2H-J).

Self-renewing murine PDGFRα⁺ CD51⁺ cells are able to transfer hematopoietic niche activity in vivo. To examine whether PDGFRα⁺ CD51⁺ cells were capable to self-renew in vivo and transfer hematopoietic activity (Mendez-Ferrer et al., 2010; Sacchetti et al., 2007), two different transplantation approaches were used to deliver single clonal PDGFRα⁺ CD51⁺ spheres derived from the BM of Nes-Gfp mice. In the first approach, single spheres were incorporated into collagen grafts and implanted under kidney capsules (FIG. 2K-L), and alternatively, spheres were implanted subcutaneously within collagen sponge (gelfoam) grafts (FIG. 2M-N). Eight weeks after transplantation, Nes-GFP⁺ cells were detected inside the grafts and in close contact with host CD45⁺ hematopoietic cells recruited in the extramedullary microenvironment (FIGS. 2L and N). By contrast, PDGFRα⁻ CD51⁺ and PDGFRα⁺ CD51⁻ spheres did not display any self-renewing Nes-GFP⁺ cells, and very few CD45⁺ hematopoietic cells were present inside the graft. The same result was further confirmed when five thousand freshly sorted Nes-GFP⁻, PDGFRα⁻ CD51⁻, PDGFRα⁻ CD51⁺ or PDGFRα⁺ CD51⁻ cells were directly transplanted (data not shown). Controls included non-transplanted kidney capsules and empty grafts without cells which only showed very rare CD45⁺ inflammatory cells. To investigate whether in vivo transplanted PDGFRα⁺ CD51⁺ cells maintained their stem cell properties, their ability to form secondary spheres was tested. Eight weeks after transplantation, grafts were collected and dissociated into single-cell suspensions. These cells were able to give rise to secondary clonal spheres (FIG. 20) that retained Nes-GFP expression (FIG. 2P), providing further proof of their self-renewing capacity. Thus, these results support the idea that HSC niche and MSC activities co-segregate in the BM.

PDGFRα and CD51 identify Nestin⁺ cells in the human fetal BM. The identification of surface markers that represent Nes-GFP⁺ cells gives an opportunity to investigate whether a similar stromal population is present in human BM. A population of human Nestin⁺ cells with similar morphology to murine cells has indeed been observed in the human adult BM (Ferraro et al., 2011) and cultured adherent BM stromal cells (Schajnovitz et al., 2011). In keeping with these results, staining of human fetal BM sections revealed the presence of elongated, pericyte-like and small rounded Nestin⁺ cells as seen in the mouse counterpart, localized in close contact with the newly formed bone/cartilage.

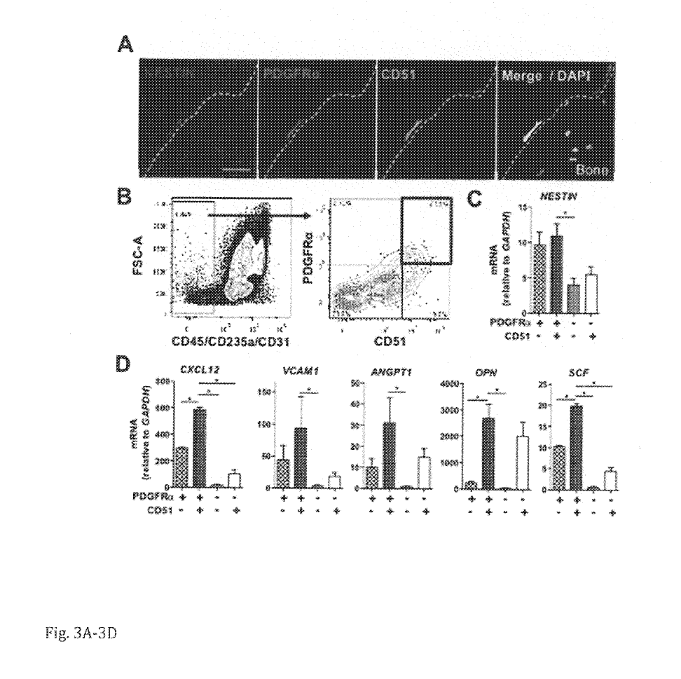

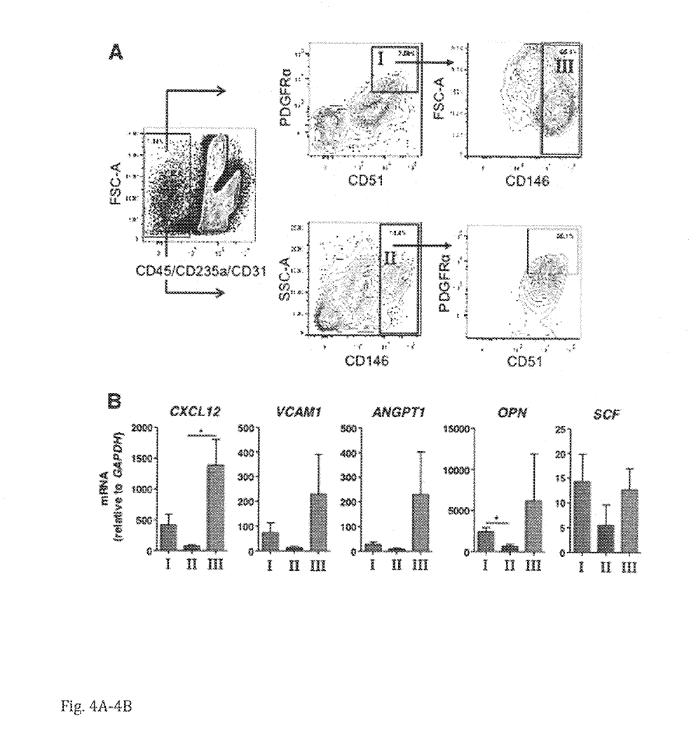

Whole mount immunofluorescence analyses for PDGFRα⁺ CD51⁺ cells revealed co-localization with Nestin⁺ cells in the human fetal bone marrow (FIG. 3A). Cell sorting of stromal cells (CD45⁻ CD235⁻a CD31⁻) expressing PDGFRα and/or CD51 revealed robust NESTIN expression in PDGFRα⁺ cells (FIGS. 3B and C). Freshly isolated human fetal PDGFRα⁺ CD51⁺ cells expressed high levels of HSC maintenance genes (CXCL12, VCAM1, ANGPT1, OPN and SCF; FIG. 3D). Since culture-expanded human CD146^{high} cells were previously shown to be highly enriched in CFU-F activity and capable of establishing the hematopoietic microenvironment in a xenotransplantation model (Sacchetti et al., 2007), CD146 expression was evaluated in the PDGFRα⁺ CD51⁺ fractions of stromal cells. An overlap was found between the two populations as ˜30% of the CD146^{high} cells also expressed PDGFRα⁺ CD51⁺, and ˜65% of PDGFRα⁺ CD51⁺ cells were also CD146^{high}, as tested in 19-20 gestation weeks (gw) human fetal bone marrow samples (FIG. 4A). Importantly, the expression of HSC maintenance genes was highly enriched in the PDGFRα⁺ CD51⁺ CD146^{high} fraction, compared to single CD146^{high} stromal cells (FIG. 4B). These results suggest that PDGFRα, CD51 and CD146 markedly enrich for HSC niche activity in the human bone marrow.

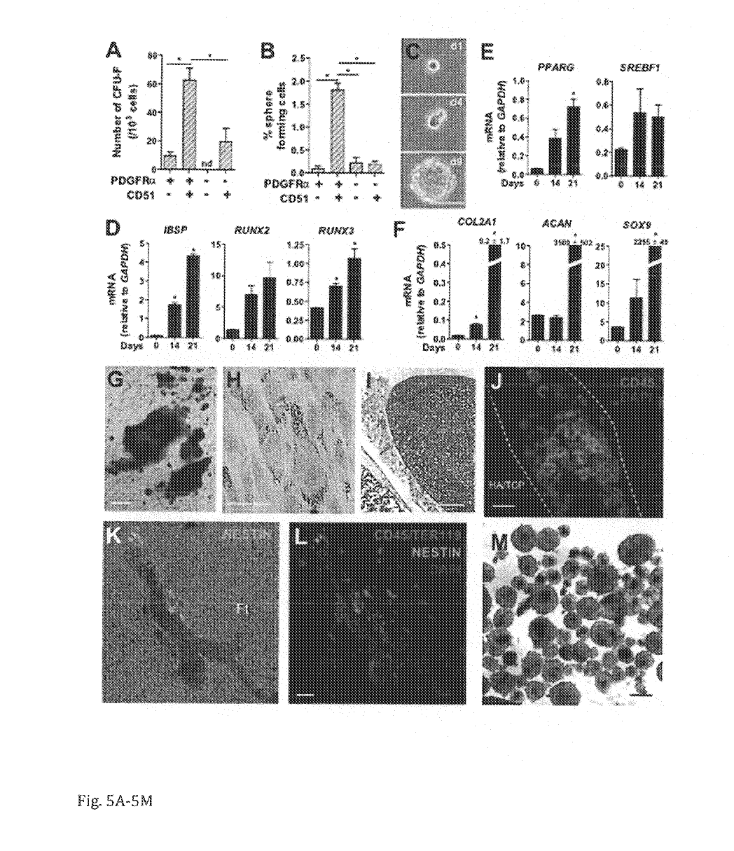

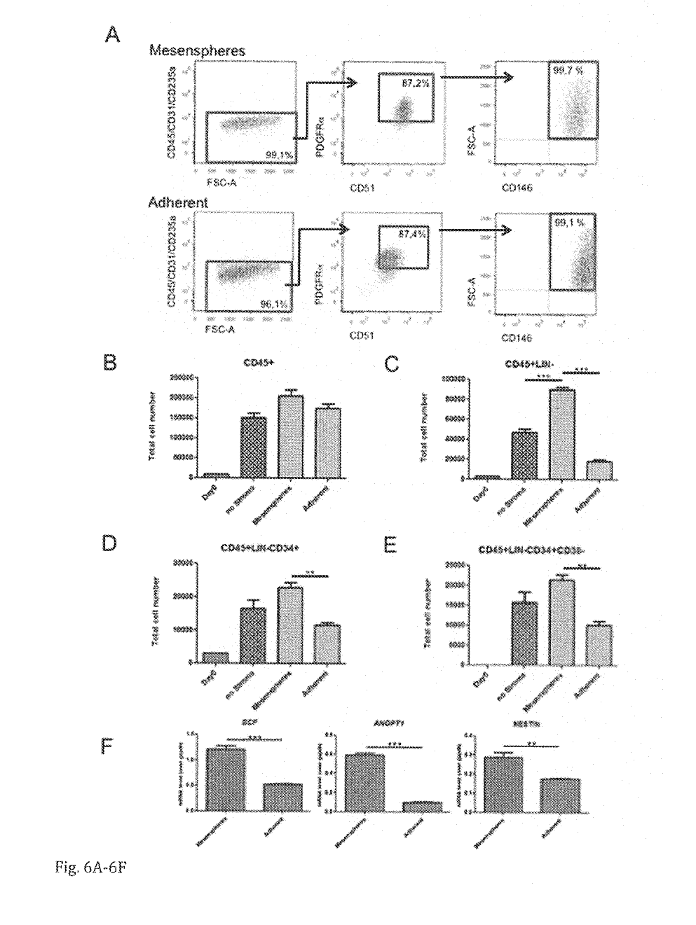

Human fetal PDGFRα+CD51+ cells are bona fide MSC: To test whether PDGFRα⁺ CD51⁺ cells exhibit features of MSCs, CFU-F content was evaluated in double- and single-positive fractions and it was found that that the highest clonogenic capacity was in PDGFRα⁺ CD51⁺ cells (FIG. 5A). Further, human PDGFRα⁺ CD51⁺ cells were able to efficiently form non-adherent primary spheres in comparison to other stromal subpopulations (FIGS. 5B and C), when plated at clonal densities using the same condition as for the murine spheres. Human clonal PDGFRα⁺ CD51⁺ spheres were able to efficiently self-renew in vitro forming secondary spheres upon dissociation that retain PDGFRα⁺ CD51⁺ and CD146^{high} expression in culture (FIG. 6A).

Fetal human PDGFRα⁺ CD51⁺ bone marrow cells were also capable of robust tri-lineage differentiation into osteoblastic (FIGS. 5D and G), adipocytic (FIGS. 5E and H) and chondrocytic (FIGS. 5F and I) mesenchymal lineages, further demonstrating their MSC identity.

HSC niche activity of human fetal PDGFRα⁺ CD51⁺ cells: To assess in vivo self-renewal, single clonal PDGFRα⁺ CD51⁺ spheres were culture-expanded, and transplanted in conjunction with hydroxyapatite/tricalcium phosphate (HA/TCP) carrier particles s.c. into immunodeficient mice. Prior to transplantation, culture-expanded cells homogeneously expressed PDGFRα and CD51 (data not shown). Eight weeks after transplantation, foci of murine hematopoietic activity was inside the graft (FIG. 5J). Since PDGFRα and CD51 epitopes are sensitive to degradation due to the decalcification process, the presence of MSC were investigated in situ by staining for human-specific anti-Nestin. Self-renewing Nestin⁺ cells were detected in the perivascular regions surrounding branching sinusoids containing murine (Ter119⁺) red blood cells (FIG. 5K-L). Consistent with their self-renewal capacity, transplanted human PDGFRα⁺ CD51⁺ cells were capable to form secondary clonal spheres in culture (FIG. 5M). By contrast, very few CD45+ hematopoietic cells were observed in the heterotopic grafts formed by non-clonally expanded and transplanted human PDGFRα⁺ CD51⁻ and PDGFRα⁻ CD51⁺ cells. Negative control grafts carrying no cells only showed the presence of fibrous connective tissue and very rare CD45⁺ cells (data not shown).

Expansion capacity of human fetal PDGFRα+CD51+CD146^{high} population: To assess the capacity of this population to expand HSC, we performed co-culture experiment with hBM CD34+ and PDGFRα⁺CD51⁺CD146^{high} cells grown as either clonal non-adherent spheres or as adherent cells. We find that the PDGFRα⁺CD51⁺CD146^{high} population grown as spheres possess a better capacity to expand HSC compared to the same population grown as adherent cells.

### Discussion

While near homogeneous populations of HSC and progenitors have been extensively isolated and characterized, the identity and role of the stromal cells regulating hematopoiesis remain largely unknown. Progress has been hampered by the limited availability of freshly isolated tissues, and the paucity of selective stromal markers and genetic tools. Common methods to isolate human MSCs have widely relied on plastic adherence and in vitro expansion of adherent cells which invariably lead to heterogeneous stromal populations whose biological and immunophenotypic properties are modulated in culture (Delorme et al., 2008; Liu et al., 2012; Sacchetti et al., 2007; Tanabe et al., 2008). Here, Nes-Gfp transgenic mice have been used which mark a highly enriched fraction of MSC that form the HSC niche (Mendez-Ferrer et al., 2010) to identify an equivalent in situ population defined by PDGFRα⁺ CD51⁺ CD45⁻ CD235a⁻ (or Ter119⁻ in mice) CD31⁻ representing a subset of Nestin⁺ cells that can be isolated prospectively in both mouse and human BM.

Although the previous studies have suggested that the two stem cell types of the BM formed a single niche, only a fraction of Nestin⁺ cells exhibits MSC activity by mesensphere or CFU-F assays (Mendez-Ferrer et al., 2010). This could be due to heterogeneity within the Nestin⁺ fraction and/or the altered cell viability following harsh isolation protocols. The fact that the frequency of Nestin⁺ cells (0.03-0.08%) is higher than that of HSCs raises the possibility that MSC and HSC maintenance properties could be conferred by distinct cells. The present studies have given more insight in this question as PDGFRα⁺ CD51⁺ stromal cells marked a subset (˜60%) of Nestin⁺ cells that enriched similarly for both HSC niche and MSC activities compared to the remaining Nestin⁺ cells. These results lend further support to the contention that these two activities co-segregate in the BM.

The results show that PDGFRα, an early development marker of a transient wave of MSC progenitors derived from neuroepithelial and neural crest lineages (Takashima et al., 2007), is a major marker for Nestin⁺ MSCs. Since neural crest stem cell-derived spheres also express Nestin (Nagoshi et al., 2008), both markers may overlap during early development. PDGFRα was recently used to isolate a perivascular population of CD45⁻ Ter119⁻ PDGFRα⁺ Sca-1⁺ cells from the adult mouse BM enriched for CFU-F activity and capable to differentiate into mesenchymal lineages (Morikawa et al., 2009). The results indicate that the vast majority (˜90%) of BM Nestin⁺ cells do not express Sca-1. Further studies are needed to clarify the difference among these subpopulations. However, there is a likely overlap between Nestin⁺ cells and a population of CD45⁻ Tie-2⁻ CD51⁺ CD105⁺ CD90⁻ cells isolated from E15.5 mouse fetal bones capable of generating heterotopic BM niche in a transplantation model (Chan et al., 2009). In addition, ˜50% of Nes-GFP⁺ cells express leptin receptor, a marker recently shown to identify BM perivascular cells producing SCF required for HSC maintenance in the BM (Ding et al., 2012) (data not shown). These observations suggest some degree of overlap between subsets of Nestin+ cells and other constituents of the HSC niche but further characterization remains to be done to tease apart the identity and function of each stromal constituents.

A major advance of the current studies is to identify a population similar to Nestin⁺ cells in the human bone marrow. PDGFRα, CD51 and CD146 in human fetal bone marrow mark a subset of stromal cells expressing Nestin that is highly enriched in CFU-F activity. Like its mouse counterpart, freshly sorted human stromal PDGFRα⁺ CD51⁺CD146^{high} cells also express high levels of HSC maintenance genes and form efficiently clonal multipotent self-renewing mesenspheres. Importantly, these cells are capable of generating heterotopically bone marrow niche in a transplantation model, whereas a subset of self-renewing perivascular cells retains Nestin expression. Previous studies have shown that human CD146^{high} bone marrow cells comprised osteoprogenitors capable of generating hematopoiesis in heterotopic bones (Sacchetti et al., 2007). Although the results indicate that CD146 is not expressed on murine Nestin⁺ cells, genome-wide expression profile of these cells was closest to that of human CD146⁺ bone marrow cells (Mendez-Ferrer et al., 2010), suggesting that CD146 may mark a stromal cell similar to murine Nestin⁺ cells. Indeed, the results show that PDGFRα⁺ CD51⁺ cells comprise a subset of CD146^{high} stromal cells further enriched for HSC niche activity in the fetal human bone marrow Immunophenotypically, most PDGFRα⁺ CD51⁺ CD146^{high} human fetal stromal cells (>90%) also express the classical MSC marker CD105 (data not shown).

Another major advance of this study is that specific culture conditions are defined for the PDGFRα⁺CD51⁺CD146^{high} population. Growing these cells as non-adherent sphere is preferred for the capacity of these cells to expand human hematopoietic stem cells.

In summary, the results demonstrate obtention of a self-renewing, multipotent population of Nestin⁺ MSCs which are an important constituent of the human fetal HSC niche. Fetal bone marrow MSCs are likely to provide an ideal stromal support for HSC expansion.

### Example 2

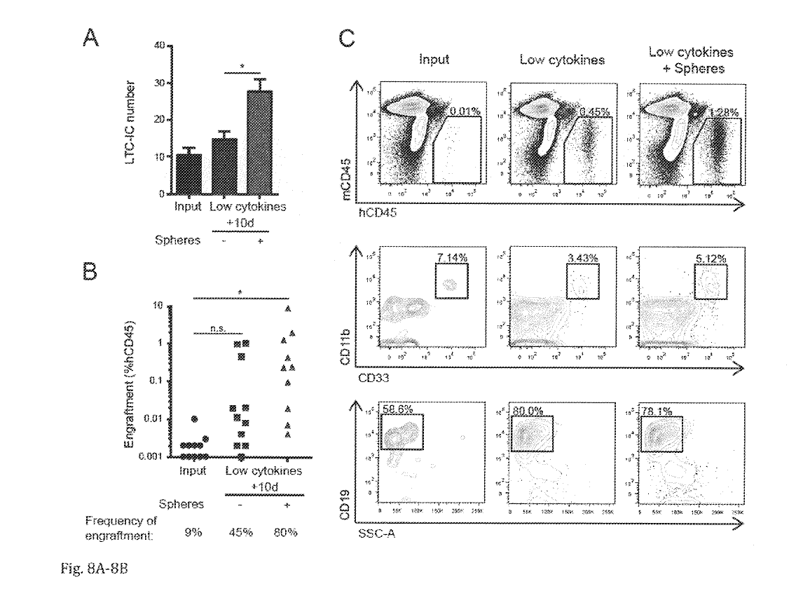

Human PDGFRα+ CD51+ mesenspheres expand HSC and progenitor cells ex vivo: To further validate the expansion of phenotypic HSC and progenitor cells, two functional assays were performed. Firstly, the frequency of long-term culture-initiating cells (LTC-IC) among Lin− CD34+ cells was quantified. Using this strategy, it was observed that the number of LTC-IC was increased by 2-fold when CD34+ cells were cultured with mesenspheres in comparison to CD34+ cells cultured with cytokines only (FIG. 8A). Second, the engraftment ability of ex vivo-expanded HSC and progenitors was analyzed. It was found that mesensphere-expanded fetal BM CD34+ cells led to a significant increase in the proportion of engrafted NSG mice 8 weeks after transplantation (80% vs 9%, *p<0.05; Fisher's exact test, FIG. 8B). By contrast, there was a non-significant trend of enhanced engraftment in the group transplanted with cells cultured with cytokines only. Furthermore mesensphere-expanded cells proved to have multilineage potential as they were able to differentiate along the myeloid and lymphoid lineages (FIG. 8C). Taken together, these data demonstrate that PDGFRα+ CD51+ mesenspheres can efficiently expand a population enriched in HSC and progenitor cells capable of multilineage engraftment.

### Materials and Methods

Long-Term Culture-Initiating Cell (LTC-IC) assay: Human CD34+ cells uncultured or cultured with cytokines for ten days in the presence or absence of mesenspheres, were plated at limiting dilution on human irradiated stroma in Myelocult media H5100 (Stem Cell Technologies) containing 10⁻³ M hydrocortisone with weekly half-media changes. After 5 weeks, the presence of LTC-IC was scored based on CFU-Cs 2 weeks after plating in MethoCult H4435 (Stem Cell Technologies). LTC-IC frequency was calculated by applying Poisson statistics using Limiting Dilution Analysis software (L-CALC, Stem Cell Technologies).

Transplantation into NSG mice: Fresh human CD34+ cells (2×10⁴) or a final culture equivalent to 2×10⁴ CD34+ input cells cultured with or without mesenspheres were transplanted via the retro-orbital route in NSG mice. NSG mice were sub-lethally irradiated (200 cGy) at least 4 h before transplantation. Bone marrow engraftment was analyzed 8 weeks post-transplantation by FACS. Mice were scored as engrafted when transplanted human cells reconstituted both myeloid and lymphoid lineages. Significance was calculated according to Fisher's exact test.

### REFERENCES

Arai, F., Hirao, A., Ohmura, M., Sato, H., Matsuoka, S., Takubo, K., Ito, K., Koh, G. Y., and Suda, T. (2004). Tie2/angiopoietin-1 signaling regulates hematopoietic stem cell quiescence in the bone marrow niche. Cell 118, 149-161.
Calvi, L. M., Adams, G. B., Weibrecht, K. W., Weber, J. M., Olson, D. P., Knight, M. C., Martin, R. P., Schipani, E., Divieti, P., Bringhurst, F. R., et al. (2003). Osteoblastic cells regulate the haematopoietic stem cell niche. Nature 425, 841-846.
Chan, C. K., Chen, C. C., Luppen, C. A., Kim, J. B., DeBoer, A. T., Wei, K., Helms, J. A., Kuo, C. J., Kraft, D. L., and Weissman, I. L. (2009). Endochondral ossification is required for haematopoietic stem-cell niche formation. Nature 457, 490-494.
Delorme, B., Ringe, J., Gallay, N., Le Vern, Y., Kerboeuf, D., Jorgensen, C., Rosset, P., Sensebe, L., Layrolle, P., Haupl, T., et al. (2008). Specific plasma membrane protein phenotype of culture-amplified and native human bone marrow mesenchymal stem cells. Blood 111, 2631-2635.
Dexter, T. M., Allen, T. D., and Lajtha, L. G. (1977). Conditions controlling the proliferation of haemopoietic stem cells in vitro. J Cell Physiol 91, 335-344.
Ding, L., Saunders, T. L., Enikolopov, G., and Morrison, S. J. (2012). Endothelial and perivascular cells maintain haematopoietic stem cells. Nature 481, 457-462.
Dominici, M., Le Blanc, K., Mueller, I., Slaper-Cortenbach, I., Marini, F., Krause, D., Deans, R., Keating, A., Prockop, D., and Horwitz, E. (2006). Minimal criteria for defining multipotent mesenchymal stromal cells. The International Society for Cellular Therapy position statement. Cytotherapy 8, 315-317.
Ferraro, F., Lymperi, S., Mendez-Ferrer, S., Saez, B., Spencer, J. A., Yeap, B. Y., Masselli, E., Graiani, G., Prezioso, L., Rizzini, E. L., et al. (2011). Diabetes impairs hematopoietic stem cell mobilization by altering niche function. Sci Transl Med 3, 104ra101.
Friedenstein, A. J., Chailakhjan, R. K., and Lalykina, K. S. (1970). The development of fibroblast colonies in monolayer cultures of guinea-pig bone marrow and spleen cells. Cell Tissue Kinet 3, 393-403.
Kiel, M. J., Yilmaz, O. H., Iwashita, T., Terhorst, C., and Morrison, S. J. (2005). SLAM family receptors distinguish hematopoietic stem and progenitor cells and reveal endothelial niches for stem cells. Cell 121, 1109-1121.
Li, Q., Yu, Y., Bischoff, J., Mulliken, J. B., and Olsen, B. R. (2003). Differential expression of CD146 in tissues and endothelial cells derived from infantile haemangioma and normal human skin. J Pathol 201, 296-302.
Liu, H., Lu, K., MacAry, P. A., Wong, K. L., Heng, A., Cao, T., and Kemeny, D. M. (2012). Soluble molecules are key in maintaining the immunomodulatory activity of murine mesenchymal stromal cells. J Cell Sci 125, 200-208.
Meirelles Lda, S., and Nardi, N. B. (2003). Murine marrow-derived mesenchymal stem cell: isolation, in vitro expansion, and characterization. Br J Haematol 123, 702-711.
Mendez-Ferrer, S., Chow, A., Merad, M., and Frenette, P. S. (2009). Circadian rhythms influence hematopoietic stem cells. Curr Opin Hematol 16, 235-242.
Mendez-Ferrer, S., Michurina, T. V., Ferraro, F., Mazloom, A. R., Macarthur, B. D., Lira, S. A., Scadden, D. T., Ma′ayan, A., Enikolopov, G. N., and Frenette, P. S. (2010). Mesenchymal and haematopoietic stem cells form a unique bone marrow niche. Nature 466, 829-834.
Mercier, F. E., Ragu, C., and Scadden, D. T. (2012). The bone marrow at the crossroads of blood and immunity. Nat Rev Immunol 12, 49-60.
Mignone, J. L., Kukekov, V., Chiang, A. S., Steindler, D., and Enikolopov, G. (2004). Neural stem and progenitor cells in nestin-GFP transgenic mice. J Comp Neurol 469, 311-324.
Morikawa, S., Mabuchi, Y., Kubota, Y., Nagai, Y., Niibe, K., Hiratsu, E., Suzuki, S., Miyauchi-Hara, C., Nagoshi, N., Sunabori, T., et al. (2009). Prospective identification, isolation, and systemic transplantation of multipotent mesenchymal stem cells in murine bone marrow. J Exp Med 206, 2483-2496.
Nagoshi, N., Shibata, S., Kubota, Y., Nakamura, M., Nagai, Y., Satoh, E., Morikawa, S., Okada, Y., Mabuchi, Y., Katoh, H., et al. (2008). Ontogeny and multipotency of neural crest-derived stem cells in mouse bone marrow, dorsal root ganglia, and whisker pad. Cell Stem Cell 2, 392-403.
Naveiras, O., Nardi, V., Wenzel, P. L., Hauschka, P. V., Fahey, F., and Daley, G. Q. (2009). Bone-marrow adipocytes as negative regulators of the haematopoietic microenvironment. Nature 460, 259-263.
Omatsu, Y., Sugiyama, T., Kohara, H., Kondoh, G., Fujii, N., Kohno, K., and Nagasawa, T. (2010). The essential functions of adipo-osteogenic progenitors as the hematopoietic stem and progenitor cell niche. Immunity 33, 387-399.
Ozerdem, U., Grako, K. A., Dahlin-Huppe, K., Monosov, E., and Stallcup, W. B. (2001). NG2 proteoglycan is expressed exclusively by mural cells during vascular morphogenesis. Dev Dyn 222, 218-227.
Phinney, D. G., Kopen, G., Isaacson, R. L., and Prockop, D. J. (1999). Plastic adherent stromal cells from the bone marrow of commonly used strains of inbred mice: variations in yield, growth, and differentiation. J Cell Biochem 72, 570-585.
Pittenger, M. F., Mackay, A. M., Beck, S. C., Jaiswal, R. K., Douglas, R., Mosca, J. D., Moorman, M. A., Simonetti, D. W., Craig, S., and Marshak, D. R. (1999). Multilineage potential of adult human mesenchymal stem cells. Science 284, 143-147.
Sacchetti, B., Funari, A., Michienzi, S., Di Cesare, S., Piersanti, S., Saggio, I., Tagliafico, E., Ferrari, S., Robey, P. G., Riminucci, M., et al. (2007). Self-renewing osteoprogenitors in bone marrow sinusoids can organize a hematopoietic microenvironment. Cell 131, 324-336.
Schajnovitz, A., Itkin, T., D'Uva, G., Kalinkovich, A., Golan, K., Ludin, A., Cohen, D., Shulman, Z., Avigdor, A., Nagler, A., et al. (2011). CXCL12 secretion by bone marrow stromal cells is dependent on cell contact and mediated by connexin-43 and connexin-45 gap junctions. Nat Immunol 12, 391-398.
Schofield, R. (1978). The relationship between the spleen colony-forming cell and the haemopoietic stem cell. Blood Cells 4, 7-25.
Sugiyama, T., Kohara, H., Noda, M., and Nagasawa, T. (2006). Maintenance of the hematopoietic stem cell pool by CXCL12-CXCR4 chemokine signaling in bone marrow stromal cell niches. Immunity 25, 977-988.
Takashima, Y., Era, T., Nakao, K., Kondo, S., Kasuga, M., Smith, A. G., and Nishikawa, S. (2007). Neuroepithelial cells supply an initial transient wave of MSC differentiation. Cell 129, 1377-1388.
Tanabe, S., Sato, Y., Suzuki, T., Suzuki, K., Nagao, T., and Yamaguchi, T. (2008). Gene expression profiling of human mesenchymal stem cells for identification of novel markers in early- and late-stage cell culture. J Biochem 144, 399-408.
Wang, L. D., and Wagers, A. J. (2011). Dynamic niches in the origination and differentiation of haematopoietic stem cells. Nat Rev Mol Cell Biol 12, 643-655.
Zhang, J., Niu, C., Ye, L., Huang, H., He, X., Tong, W. G., Ross, J., Haug, J., Johnson, T., Feng, J. Q., et al. (2003). Identification of the haematopoietic stem cell niche and control of the niche size. Nature 425, 836-841.
Bianco, P., Kuznetsov, S. A., Riminucci, M., and Gehron Robey, P. (2006). Postnatal skeletal stem cells. Methods Enzymol 419, 117-148.
Kawamoto, T. (2003). Use of a new adhesive film for the preparation of multi-purpose fresh-frozen sections from hard tissues, whole-animals, insects and plants. Arch Histol Cytol 66, 123-143.
Kuznetsov, S. A., Krebsbach, P. H., Satomura, K., Kerr, J., Riminucci, M., Benayahu, D., and Robey, P. G. (1997). Single-colony derived strains of human marrow stromal fibroblasts form bone after transplantation in vivo. J Bone Miner Res 12, 1335-1347.

1. A method of obtaining a population of stem cells comprising identifying PDGFRα⁺ CD51⁺ cells, or PDGFRα⁺ CD51⁺ CD146⁺ cells, in a heterogeneous population of cells, and recovering the PDGFRα⁺ CD51⁺ cells, or PDGFRα⁺ CD51⁺ CD146⁺ cells, so as to obtain the population of stem cells.

2. The method of claim 1, wherein recovering the PDGFRα⁺ CD51⁺ cells comprises separating the PDGFRα+ CD51+ cells from the heterogeneous population of cells using an antibody, or antigen-binding fragment thereof, directed against PDGFRα and/or using an antibody, or antigen-binding fragment thereof, directed against CD51, or wherein recovering the PDGFRα⁺ CD51⁺ CD146⁺ cells comprises separating the PDGFRα⁺ CD51⁺ CD146⁺ cells from the heterogeneous population of cells using an antibody, or antigen-binding fragment thereof, directed against PDGFRα and/or using an antibody, or antigen-binding fragment thereof, directed against CD51, and/or using an antibody, or antigen-binding fragment thereof, directed against CD146.

3. The method of claim 1, wherein the heterogenous population of cells is a population of bone marrow cells.

4. The method of claim 1, wherein the population of stem cells is a population of mesenchymal stem cells.

5. The method of claim 1, further comprising recovering CD105+ cells from the PDGFRα⁺ CD51⁺ population of stem cells or from the PDGFRα⁺ CD51⁺ CD146⁺ population of stem cells.

6-8. (canceled)

9. The method of claim 1, further comprising expanding the population of PDGFRα⁺ CD51⁺ stem cells, or PDGFRα⁺ CD51⁺ CD146⁺ stem cells, in culture.

10. The method of claim 1, wherein the stem cells are human stem cells.

11. The method of claim 1, wherein the PDGFRα⁺ CD51⁺ CD146⁺ cells are PDGFRα⁺ CD51⁺ CD146^{high}.

12. (canceled)

13. The method of claim 1, further comprising lysing red series cells in the heterogenous population of cells prior to recovering the PDGFRα⁺ CD51⁺ cells, or PDGFRα⁺ CD51⁺ CD146⁺ cells.

14. An isolated population of PDGFRα⁺ CD51⁺ mesenchymal stem cells, wherein the population is 25% or greater PDGFRα⁺ CD51⁺ cells or an isolated population of PDGFRα⁺ CD51⁺ CD146⁺ mesenchymal stem cells, wherein the population is 25% or greater PDGFRα⁺ CD51⁺ CD146⁺ cells.

15-16. (canceled)

17. The population of claim 14, having CFU-F activity and/or clonal self-renew sphere formation activity.

18-19. (canceled)

20. The population of claim 14, wherein the or PDGFRα⁺ CD51⁺ CD146⁺ cells are or PDGFRα⁺ CD51⁺ CD146^{high} cells.

21. The population of claim 14, wherein the PDGFRα⁺ CD51⁺ cells are also CD105⁺ and/or CD45−.

22-26. (canceled)

27. A composition comprising the population of claim 14 and a carrier.

28-29. (canceled)

30. A method comprising administering an amount of the population of stem cells of claim 14 to a subject in an amount effective to confer stem cell activity on a subject.

31. The method of claim 30, wherein the amount is effective to confer hematopoietic activity.

32. A method of treating a subject in need of enhanced hematopoietic activity comprising administering an amount of the population of stem cells obtained by the method of claim 1 to the subject in a manner effective to confer enhanced hematopoietic activity on a subject.

33. The method of claim 32, wherein human PDGFRα+ CD51+ mesenspheres are administered.

34. A method of expanding a population of HSC or progenitor cells comprising co-culturing the cells with PDGFRα+ CD51+ mesenspheres in an amount sufficient to efficiently expand the population of HSC or progenitor cells.

35. The method of claim 34, wherein the HSC or progenitor cells are CD34+ cells.

36. (canceled)


SEQUENCE LISTING (56 sequences)

SEQ ID NO: 1 - DNA, 20 bp, Artificial Sequence
  PRIMER DIRECTED TO HUMAN GADPH
tctgctcctcctgttcgaca

SEQ ID NO: 2 - DNA, 19 bp, Artificial Sequence
  PRIMER DIRECTED TO HUMAN GADPH
aaaagcagccctggtgacc

SEQ ID NO: 3 - DNA, 21 bp, Artificial Sequence
  PRIMER DIRECTED TO HUMAN CXCL12
tgggctcctactgtaagggtt

SEQ ID NO: 4 - DNA, 20 bp, Artificial Sequence
  PRIMER DIRECTED TO HUMAN CXCL12
ttgacccgaagctaaagtgg

SEQ ID NO: 5 - DNA, 20 bp, Artificial Sequence
  PRIMER DIRECTED TO HUMAN VCAM1
gtctccaatctgagcagcaa

SEQ ID NO: 6 - DNA, 20 bp, Artificial Sequence
  PRIMER DIRECTED TO HUMAN VCAM1
tgaggatggaagattctgga

SEQ ID NO: 7 - DNA, 20 bp, Artificial Sequence
  PRIMER DIRECTED TO HUAMN ANGPT1
gccatctccgacttcatgtt

SEQ ID NO: 8 - DNA, 20 bp, Artificial Sequence
  PRIMER DIRECTED TO HUMAN ANGPT1
ctgcagagagatgctccaca

SEQ ID NO: 9 - DNA, 20 bp, Artificial Sequence
  PRIMER DIRECTED TO HUMAN OPN
agatgggtcagggtttagcc

SEQ ID NO: 10 - DNA, 20 bp, Artificial Sequence
  PRIMER DIRECTED TO HUMAN OPN
catcacctgtgccataccag

SEQ ID NO: 11 - DNA, 23 bp, Artificial Sequence
  PRIMER DIRECTED TO HUMAN SCF
ccatctcgcttatccaacaatga

SEQ ID NO: 12 - DNA, 23 bp, Artificial Sequence
  PRIMER DIRECTED TO HUMAN SCF
aatcctctcgtcaaaactgaagg

SEQ ID NO: 13 - DNA, 19 bp, Artificial Sequence
  PRIMER DIRECTED TO HUMAN NESTIN
gggagttctcagcctccag

SEQ ID NO: 14 - DNA, 20 bp, Artificial Sequence
  PRIMER DIRECTED TO HUMAN NESTIN
ggagaaacagggcctacaga

SEQ ID NO: 15 - DNA, 23 bp, Artificial Sequence
  PRIMER DIRECTED TO HUMAN IBSP
tgaagtctcctcttcttcctcct

SEQ ID NO: 16 - DNA, 20 bp, Artificial Sequence
  PRIMER DIRECTED TO HUMAN IBSP
aaacgatttccagttcaggg

SEQ ID NO: 17 - DNA, 20 bp, Artificial Sequence
  PRIMER DIRECTED TO HUMAN RUNX2
atactgggatgaggaatgcg

SEQ ID NO: 18 - DNA, 20 bp, Artificial Sequence
  PRIMER DIRECTED TO HUMAN RUNX2
acagtagatggacctcggga

SEQ ID NO: 19 - DNA, 20 bp, Artificial Sequence
  PRIMER DIRECTED TO HUMAN RUNX3
gtctggtcctccagcttctg

SEQ ID NO: 20 - DNA, 19 bp, Artificial Sequence
  PRIMER DIRECTED TO HUMAN RUNX3
ctgtgttcaccaaccccac

SEQ ID NO: 21 - DNA, 20 bp, Artificial Sequence
  PRIMER DIRECTED TO HUMAN PPARG
gagagatccacggagctgat

SEQ ID NO: 22 - DNA, 20 bp, Artificial Sequence
  PRIMER DIRECTED TO HUMAN PPARG
aggccattttgtcaaacgag

SEQ ID NO: 23 - DNA, 17 bp, Artificial Sequence
  PRIMER DIRECTED TO HUMAN SREBF1
gttggccctacccctcc

SEQ ID NO: 24 - DNA, 17 bp, Artificial Sequence
  PRIMER DIRECTED TO HUMAN SREBF1
cttcagcgaggcggctt

SEQ ID NO: 25 - DNA, 20 bp, Artificial Sequence
  PRIMER DIRECTED TO HUMAN COL2A1
tttctgtccctttggtcctg

SEQ ID NO: 26 - DNA, 19 bp, Artificial Sequence
  PRIMER DIRECTED TO HUMAN COL2A1
gtgagccatgattcgcctc

SEQ ID NO: 27 - DNA, 20 bp, Artificial Sequence
  PRIMER DIRECTED TO HUMAN ACAN
gcgagttgtcatggtctgaa

SEQ ID NO: 28 - DNA, 20 bp, Artificial Sequence
  PRIMER DIRECTED TO HUMAN ACAN
ttcttggagaagggagtcca

SEQ ID NO: 29 - DNA, 20 bp, Artificial Sequence
  PRIMER DIRECTED TO HUMAN SOX9
gtaatccgggtggtccttct

SEQ ID NO: 30 - DNA, 17 bp, Artificial Sequence
  PRIMER DIRECTED TO HUMAN SOX9
gacgctgggcaagctct

SEQ ID NO: 31 - DNA, 20 bp, Artificial Sequence
  PRIMER DIRECTED TO MOPUSE GAPDH
tgtgtccgtcgtggatctga

SEQ ID NO: 32 - DNA, 21 bp, Artificial Sequence
  PRIMER DIRECTED TO MOUSE GAPDH
cctgcttcaccaccttcttga

SEQ ID NO: 33 - DNA, 17 bp, Artificial Sequence
  PRIMER DIRECTED TO MOUSE CXCL12
cgccaaggtcgtcgccg

SEQ ID NO: 34 - DNA, 19 bp, Artificial Sequence
  PRIMER DIRECTED TO MOUSE CXCL12
ttggctctggcgatgtggc

SEQ ID NO: 35 - DNA, 22 bp, Artificial Sequence
  PRIMER DIRECTED TO MOUSE VCAM1
gacctgttccagcgagggtcta

SEQ ID NO: 36 - DNA, 26 bp, Artificial Sequence
  PRIMER DIRECTED TO MOUSE VCAM1
cttccatcctcatagcaattaaggtg

SEQ ID NO: 37 - DNA, 23 bp, Artificial Sequence
  PRIMER DIRECTED TO MOUSE ANGPT1
ctcgtcagacattcatcatccag

SEQ ID NO: 38 - DNA, 22 bp, Artificial Sequence
  PRIMER DIRECTED TO MOUSE ANGPT1
caccttctttagtgcaaaggct

SEQ ID NO: 39 - DNA, 22 bp, Artificial Sequence
  PRIMER DIRECTED TO MOUSE OPN
tccctcgatgtcatccctgttg

SEQ ID NO: 40 - DNA, 22 bp, Artificial Sequence
  PRIMER DIRECTED TO MOUSE OPN
ggcactctcctggctctctttg

SEQ ID NO: 41 - DNA, 19 bp, Artificial Sequence
  PRIMER DIRECTED TO MOUSE SCF
ccctgaagactcgggccta

SEQ ID NO: 42 - DNA, 23 bp, Artificial Sequence
  PRIMER DIRECTED TO MOUSE SCF
caattacaagcgaaatgagagcc

SEQ ID NO: 43 - DNA, 21 bp, Artificial Sequence
  PRIMER DIRECTED TO MOUSE NESTIN
gctggaacagagattggaagg

SEQ ID NO: 44 - DNA, 21 bp, Artificial Sequence
  PRIMER DIRECTED TO MOUSE NESTIN
ccaggatctgagcgatctgac

SEQ ID NO: 45 - DNA, 21 bp, Artificial Sequence
  PRIMER DIRECTED TO  MOUSE GPNMB
ccccaagcacagacttttgag

SEQ ID NO: 46 - DNA, 20 bp, Artificial Sequence
  PRIMER DIRECTED TO MOUSE GPNMB
gctttctgcatctccagcct

SEQ ID NO: 47 - DNA, 22 bp, Artificial Sequence
  PRIMER DIRECTED TO MOUSE OGN
accataacgacctggaatctgt

SEQ ID NO: 48 - DNA, 21 bp, Artificial Sequence
  PRIMER DIRECTED TO MOUSE OGN
aacgagtgtcattagccttgc

SEQ ID NO: 49 - DNA, 20 bp, Artificial Sequence
  PRIMER DIRECTED TO MOUSE SP7
atggcgtcctctctgcttga

SEQ ID NO: 50 - DNA, 20 bp, Artificial Sequence
  PRIMER DIRECTED TO MOUSE SP7
gaagggtgggtagtcatttg

SEQ ID NO: 51 - DNA, 20 bp, Artificial Sequence
  PRIMER DIRECTED TO MOUSE PPARG
accactcgcattcctttgac

SEQ ID NO: 52 - DNA, 20 bp, Artificial Sequence
  PRIMER DIRECTED TO MOUSE PPARG
tgggtcagctcttgtgaatg

SEQ ID NO: 53 - DNA, 24 bp, Artificial Sequence
  PRIMER DIRECTED TO MOUSE CFD
tgcatcaactcagagtgtcaatca

SEQ ID NO: 54 - DNA, 19 bp, Artificial Sequence
  PRIMER DIRECTED TO MOUSE CFD
tgcgcagattgcaggttgt

SEQ ID NO: 55 - DNA, 21 bp, Artificial Sequence
  PRIMER DIRECTED TO MOUSE ACAN
cacgctacaccctggactttg

SEQ ID NO: 56 - DNA, 21 bp, Artificial Sequence
  PRIMER DIRECTED TO MOUSE ACAN
ccatctcctcagcgaagcagt